SEQUENCE LISTING (28 sequences)

SEQ ID NO: 1 - protein, 462 aa, Homo sapiens
MARVLGAPVALGLWSLCWSLAIATPLPPTSAHGNVAEGETKPDPDVTERCSDGWSFDATTLDDNGTMLFFKGEFVWKSHKWDRELISERWKNFPSPVDAAFRQGHNSVFLIKGDKVWVYPPEKKEKGYPKLLQDEFPGIPSPLDAAVECHRGECQAEGVLFFQGDREWFWDLATGTMKERSWPAVGNCSSALRWLGRYYCFQGNQFLRFDPVRGEVPPRYPRDVRDYFMPCPGRGHGHRNGTGHGNSTHHGPEYMRCSPHLVLSALTSDNHGATYAFSGTHYWRLDTSRDGWHSWPIAHQWPQGPSAVDAAFSWEEKLYLVQGTQVYVFLTKGGYTLVSGYPKRLEKEVGTPHGIILDSVDAAFICPGSSRLHIMAGRRLWWLDLKSGAQATWTELPWPHEKVDGALCMEKSLGPNSCSANGPGLYLIHGPNLYCYSDVEKLNAAKALPQPQNVTSLLGCTH

SEQ ID NO: 2 - protein, 201 aa, Homo sapiens
MALSWVLTVLSLLPLLEAQIPLCANLVPVPITNATLDRITGKWFYIASAFRNEEYNKSVQEIQATFFYFTPNKTEDTIFLREYQTRQDQCIYNTTYLNVQRENGTISRYVGGQEHFAHLLILRDTKTYMLAFDVNDEKNWGLSVYADKPETTKEQLGEFYEALDCLRIPKSDVVYTDWKKDKCEPLEKQHEKERKQEEGES

SEQ ID NO: 3 - protein, 201 aa, Homo sapiens
MALSWVLTVLSLLPLLEAQIPLCANLVPVPITNATLDRITGKWFYIASAFRNEEYNKSVQEIQATFFYFTPNKTEDTIFLREYQTRQNQCFYNSSYLNVQRENGTVSRYEGGREHVAHLLFLRDTKTLMFGSYLDDEKNWGLSFYADKPETTKEQLGEFYEALDCLCIPRSDVMYTDWKKDKCEPLEKQHEKERKQEEGES

SEQ ID NO: 4 - protein, 418 aa, Homo sapiens
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 5 - protein, 500 aa, Homo sapiens
MASRLTLLTLLLLLLAGDRASSNPNATSSSSQDPESLQDRGEGKVATTVISKMLFVEPILEVSSLPTTNSTTNSATKITANTTDEPTTQPTTEPTTQPTIQPTQPTTQLPTDSPTQPTTGSFCPGPVTLCSDLESHSTEAVLGDALVDFSLKLYHAFSAMKKVETNMAFSPFSIASLLTQVLLGAGENTKTNLESILSYPKDFTCVHQALKGFTTKGVTSVSQIFHSPDLAIRDTFVNASRTLYSSSPRVLSNNSDANLELINTWVAKNTNNKISRLLDSLPSDTRLVLLNAIYLSAKWKTTFDPKKTRMEPFHFKNSVIKVPMMNSKKYPVAHFIDQTLKAKVGQLQLSHNLSLVILVPQNLKHRLEDMEQALSPSVFKAIMEKLEMSKFQPTLLTLPRIKVTTSQDMLSIMEKLEFFDFSYDLNLCGLTEDPDLQVSAMQHQTVLELTETGVEAAAASAISVARTLLVFEVQQPFLFVLWDQQHKFPVFMGRVYDPRA

SEQ ID NO: 6 - protein, 330 aa, Homo sapiens [sequence omitted]

SEQ ID NO: 7 - protein, 1474 aa, Homo sapiens
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDNEDCINRHNVYINGITYTPVSSTNEKDMYSFLEDMGLKAFTNSKIRKPKMCPQLQQYEMHGPEGLRVGFYESDVMGRGHARLVHVEEPHTETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEIKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSNHVSRTEVSSNHVLIYLDKVSNQTLSLFFTVLQDVPVRDLKPAIVKVYDYYETDEFAIAEYNAPCSKDLGNA

SEQ ID NO: 8 - protein, 406 aa, Homo sapiens
MSALGAVIALLLWGQLFAVDSGNDVTDIADDGCPKPPEIAHGYVEHSVRYQCKNYYKLRTEGDGVYTLNDKKQWINKAVGDKLPECEADDGCPKPPEIAHGYVEHSVRYQCKNYYKLRTEGDGVYTLNNEKQWINKAVGDKLPECEAVCGKPKNPANPVQRILGGHLDAKGSFPWQAKMVSHHNLTTGATLINEQWLLTTAKNLFLNHSENATAKDIAPTLTLYVGKKQLVEIEKVVLHPNYSQVDIGLIKLKQKVSVNERVMPICLPSKDYAEVGRVGYVSGWGRNANFKFTDHLKYVMLPVADQDQCIRHYEGSTVPEKKTPKSPVGVQPILNEHTFCAGMSKYQEDTCYGDAGSAFAVHDLEEDTWYATGILSFDKSCAVAEYGVYVKVTSIQDWVQKTIAEN

SEQ ID NO: 9 - protein, 453 aa, Homo sapiens
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 10 - protein, 525 aa, Homo sapiens
MKALIAALLLITLQYSCAVSPTDCSAVEPEAEKALDLINKRRRDGYLFQLLRIADAHLDRVENTTVYYLVLDVQESDCSVLSRKYWNDCEPPDSRRPSEIVIGQCKVIATRHSHESQDLRVIDFNCTTSSVSSALANTKDSPVLIDFFEDTERYRKQANKALEKYKEENDDFASFRVDRIERVARVRGGEGTGYFVDFSVRNCPRHHFPRHPNVFGFCRADLFYDVEALDLESPKNLVINCEVFDPQEHENINGVPPHLGHPFHWGGHERSSTTKPPFKPHGSRDHHHPHKPHEHGPPPPPDERDHSHGPPLPQGPPPLLPMSCSSCQHATFGTNGAQRHSHNNNSSDLHPHKHHSHEQHPHGHHPHAHHPHEHDTHRQHPHGHHPHGHHPHGHHPHGHHPHGHHPHCHDFQDYGPCDPPPHNQGHCCHGHGPPPGHLRRRGPGKGPRPFHCRQIGSVYRLPPLRKGEVLPLPEANFPSFPLPHHKHPLKPDNQPFPQSVSESCPGKFKSGFPQVSMFFTHTFPK

SEQ ID NO: 11 - protein, 1691 aa, Homo sapiens
MASWTSPWWVLIGMVFMHSPLPQTTAEKSPGAYFLPEFALSPQGSFLEDTTGEQFLTYRYDDQTSRNTRSDEDKDGNWDAWGDWSDCSRTCGGGASYSLRRCLTGRNCEGQNIRYKTCSNHDCPPDAEDFRAQQCSAYNDVQYQGHYYEWLPRYNDPAAPCALKCHAQGQNLVVELAPKVLDGTRCNTDSLDMCISGICQAVGCDRQLGSNAKEDNCGVCAGDGSTCRLVRGQSKSHVSPEKREENVIAVPLGSRSVRITVKGPAHLFIESKTLQGSKGEHSFNSPGVFLVENTTVEFQRGSERQTFKIPGPLMADFIFKTRYTAAKDSVVQFFFYQPISHQWRQTDFFPCTVTCGGGYQLNSAECVDIRLKRVVPDHYCHYYPENVKPKPKLKECSMDPCPSSDGFKEIMPYDHFQPLPRWEHNPWTACSVSCGGGIQRRSFVCVEESMHGEILQVEEWKCMYAPKPKVMQTCNLFDCPKWIAMEWSQCTVTCGRGLRYRVVLCINHRGEHVGGCNPQLKLHIKEECVIPIPCYKPKEKSPVEAKLPWLKQAQELEETRIATEEPTFIPEPWSACSTTCGPGVQVREVKCRVLLTFTQTETELPEEECEGPKLPTERPCLLEACDESPASRELDIPLPEDSETTYDWEYAGFTPCTATCVGGHQEAIAVCLHIQTQQTVNDSLCDMVHRPPAMSQACNTEPCPPRWHVGSWGPCSATCGVGIQTRDVYCLHPGETPAPPEECRDEKPHALQACNQFDCPPGWHIEEWQQCSRTCGGGTQNRRVTCRQLLTDGSFLNLSDELCQGPKASSHKSCARTDCPPHLAVGDWSKCSVSCGVGIQRRKQVCQRLAAKGRRIPLSEMMCRDLPGLPLVRSCQMPECSKIKSEMKTKLGEQGPQILSVQRVYIQTREEKRINLTIGSRAYLLPNTSVIIKCPVRRFQKSLIQWEKDGRCLQNSKRLGITKSGSLKIHGLAAPDIGVYRCIAGSAQETVVLKLIGTDNRLIARPALREPMREYPGMDHSEANSLGVTWHKMRQMWNNKNDLYLDDDHISNQPFLRALLGHCSNSAGSTNSWELKNKQFEAAVKQGAYSMDTAQFDELIRNMSQLMETGEVSDDLASQLIYQLVAELAKAQPTHMQWRGIQEETPPAAQLRGETGSVSQSSHAKNSGKLTFKPKGPVLMRQSQPPSISFNKTINSRIGNTVYITKRTEVINILCDLITPSEATYTWTKDGTLLQPSVKIILDGTGKIQIQNPTRKEQGIYECSVANHLGSDVESSSVLYAEAPVILSVERNITKPEHNHLSVVVGGIVEAALGANVTIRCPVKGVPQPNITWLKRGGSLSGNVSLLFNGSLLLQNVSLENEGTYVCIATNALGKAVATSVLHLLERRWPESRIVFLQGHKKYILQATNTRTNSNDPTGEPPPQEPFWEPGNWSHCSATCGHLGARIQRPQCVMANGQEVSEALCDHLQKPLAGFEPCNIRDCPARWFTSVWSQCSVSCGEGYHSRQVTCKRTKANGTVQVVSPRACAPKDRPLGRKPCFGHPCVQWEPGNRCPGRCMGRAVRMQQRHTACQHNSSDSNCDDRKRPTLRRNCTSGACDVCWHTGPWKPCTAACGRGFQSRKVDCIHTRSCKPVAKRHCVQKKKPISWRHCLGPSCDRDCTDTTHYCMFVKHLNLCSLDRYKQRCCQSCQEG

SEQ ID NO: 12 - protein, 22 aa, Homo sapiens
CSDGWSFDATTLDDNGTMLFFK

SEQ ID NO: 13 - protein, 24 aa, Homo sapiens
SVQEIQATFFYFTPNKTEDTIFLR

SEQ ID NO: 14 - protein, 16 aa, Homo sapiens
YLGNATAIFFLPDEGK

SEQ ID NO: 15 - protein, 19 aa, Homo sapiens
VLSNNSDANLELINTWVAK

SEQ ID NO: 16 - protein, 9 aa, Homo sapiens
EEQYNSTYR

SEQ ID NO: 17 - protein, 17 aa, Homo sapiens
NGTGHGNSTHHGPEYMR

SEQ ID NO: 18 - protein, 19 aa, Homo sapiens
VSNQTLSLFFTVLQDVPVR

SEQ ID NO: 19 - protein, 30 aa, Homo sapiens
QLAHQSNSTNIFFSPVSIATAFAMLSLGTK

SEQ ID NO: 20 - protein, 32 aa, Homo sapiens
SLGNVNFTVSAEALESQELCGTEVPSVPEHGR

SEQ ID NO: 21 - protein, 24 aa, Homo sapiens
MVSHHNLTTGATLINEQWLLTTAK

SEQ ID NO: 22 - protein, 21 aa, Homo sapiens
GLTFQQNASSMCVPDQDTAIR

SEQ ID NO: 23 - protein, 16 aa, Homo sapiens
SVQEIQATFFYFTPNK

SEQ ID NO: 24 - protein, 22 aa, Homo sapiens
GCVLLSYLNETVTVSASLESVR

SEQ ID NO: 25 - protein, 14 aa, Homo sapiens
SSTTKPPFKPHGSR

SEQ ID NO: 26 - protein, 8 aa, Homo sapiens
IILDGTGK

SEQ ID NO: 27 - protein, 7 aa, synthetic construct
ECDMHYK

SEQ ID NO: 28 - protein, 7 aa, synthetic construct
  SITE 2: note = acetamide-modified Cysteine
ECDMHYK

(19) United States
(12) Patent Application Publication
(10) Pub. No.: US 2025/0087363 A1
(43) Pub. Date: Mar. 13, 2025

(54) PREDICTING SARCOMA TREATMENT RESPONSE USING TARGETED QUANTIFICATION OF SITE-SPECIFIC PROTEIN GLYCOSYLATION

(71) Applicant: VENN BIOSCIENCES CORPORATION, South San Francisco, CA (US)

(72) Inventors: Chad Eagle PICKERING, South San Francisco, CA (US); Daniel SERIE, South San Francisco, CA (US); Gege XU, South San Francisco, CA (US)

(21) Appl. No.: 18/711,191

(22) PCT Filed: Nov. 22, 2022
(86) PCT No.: PCT/IB2022/061293

§ 371 Date: May 17, 2024

Related U.S. Application Data
(60) Provisional application No. 63/282,168, filed on Nov. 22, 2021.

(51) Int. Cl. G16H 50/30; G16B 15/00; G16H 20/17

(52) U.S. Cl. CPC G16H 50/30; G16B 15/00; G16H 20/17

(57) ABSTRACT

A method and system for managing a treatment for a subject diagnosed with a sarcoma disease state. Peptide structure data corresponding to a biological sample obtained from the subject is received. A response score that predicts a likelihood of responsiveness to the treatment is computed using quantification data identified from the peptide structure data for a set of peptide structures. The set of peptide structures includes at least one peptide structure identified from a plurality of peptide structures listed in Table 1. The plurality of peptide structures is listed in Table 1 with respect to relative significance to a survival for the sarcoma disease state. A treatment response output is generated based on the response score.

### CROSS-REFERENCE TO RELATED APPLICATIONS

This application claims priority to and the benefit of U.S. Application No. 63/282,168, filed Nov. 22, 2021, the contents of which are incorporated herein in its entirety.

### FIELD

The present disclosure generally relates to methods and systems for analyzing peptide structures for managing treatment of sarcoma. More particularly, the present disclosure relates to analyzing quantification data for a set of peptide structures detected in a biological sample obtained from a subject diagnosed with sarcoma for use in predicting a responsiveness of the subject to treatment (e.g., an immunotherapy).

### BACKGROUND

Protein glycosylation and other post-translational modifications play vital roles in virtually all aspects of human physiology. Unsurprisingly, faulty or altered protein glycosylation often accompanies various disease states. The identification of aberrant glycosylation provides opportunities for early detection, intervention, and treatment of affected subjects. Current biomarker identification methods, such as those developed in the fields of proteomics and genomics, can be used to detect indicators of certain diseases, such as cancer, and to differentiate certain types of cancer from other, non-cancerous diseases. However, the use of glycoproteomic analyses has not previously been used to successfully manage treatment of a subject.

Glycoprotein analysis is fraught with challenges on several levels. For example, a single glycan composition in a glycopeptide can contain a large number of isomeric structures due to different glycosidic linkages, branching patterns, and/or multiple monosaccharides having the same mass. In addition, the presence of multiple glycans that share the same peptide backbone can lead to assay signals from various glycoforms, lowering their individual abundances compared to aglycosylated peptides. Accordingly, the development of algorithms that can identify glycan structures on peptide fragments remains elusive.

In light of the above, there is a desire for improved analytical methods that involve site-specific analysis of glycoproteins to obtain information about protein glycosylation patterns, which can in turn provide quantitative information that can be used to manage the treatment of a subject diagnosed with a particular disease or condition. For example, there is a desire to use site-specific analysis of glycoproteins to determine whether a subject diagnosed with sarcoma is likely to respond to treatment or not. Thus, it may be desirable to have methods and systems capable of addressing one or more of the above-identified issues.

### SUMMARY

In one or more embodiments, a method is provided for managing a treatment of a subject diagnosed with a sarcoma disease state. Peptide structure data corresponding to a biological sample obtained from the subject is received. A response score that predicts a likelihood of responsiveness to the treatment is predicted using quantification data identified from the peptide structure data for a set of peptide structures. The set of peptide structures includes at least one peptide structure identified from a plurality of peptide structures listed in Table 1. The plurality of peptide structures is listed in Table 1 with respect to relative significance to a survival for the sarcoma disease state. A treatment response output is generated based on the response score.

In one or more embodiments, a method is provided for building a final model to predict treatment responsiveness for a subject diagnosed with a sarcoma disease state. Sample data is received for a panel of peptide structures for a plurality of sample subjects diagnosed with the sarcoma disease state. The sample data comprises quantification data for the panel of peptide structures. Survival information is received for the plurality of sample subjects. Based on the sample data and the survival information, an initial group of peptide structures that are associated with survival of the sarcoma disease state is identified. The initial group of peptide structures includes at least 3 peptide structures of a plurality of peptide structures identified in Table 1. A plurality of models is built using different subsets of the initial group of peptide structures. The final model is selected from the plurality of models for use in predicting the treatment responsiveness for the treatment to sarcoma.

In one or more embodiments, a method is provided for treating sarcoma in a patient. Peptide structure data corresponding to a biological sample obtained from the patient is received. A response score that predicts a likelihood of responsiveness to a treatment is computed using quantification data identified from the peptide structure data for a set of peptide structures. The set of peptide structures includes at least one peptide structure identified from a plurality of peptide structures listed in Table 1. The plurality of peptide structures is listed in Table 1 with respect to relative significance to a survival for the sarcoma disease state. A determination is made as to whether the subject is a likely responder or a likely non-responder for the treatment based on the response score. A therapeutic dosage of the treatment is administered to the patient if the subject is determined to be the likely responder.

In one or more embodiments, a composition comprises at least one of peptide structures PS-1 to PS-22 identified in Table 1.

In one or more embodiments, a composition comprises a peptide structure or a product ion. The peptide structure or product ion comprises an amino acid sequence having at least 90% sequence identity to any one of SEQ ID NOS: 12-26, corresponding to peptide structures PS-1 to PS-22 in Table 1. The product ion is selected as one from a group consisting of product ions identified in Table 2 including product ions falling within an identified m/z range.

In one or more embodiments, a composition comprises a glycopeptide structure selected as one from a group consisting of peptide structures PS-1 to PS-22 identified in Table 1. The glycopeptide structure comprises an amino acid peptide sequence identified in Table 3 as corresponding to the glycopeptide structure. The glycopeptide structure comprises a glycan structure identified in Table 5 as corresponding to the glycopeptide structure in which the glycan structure is linked to a residue of the amino acid peptide sequence at a corresponding position identified in Table 1. The glycan structure has a glycan composition.

In one or more embodiments, a composition comprises a peptide structure selected as one from a plurality of peptide structures identified in Table 1. The peptide structure has a monoisotopic mass identified as corresponding to the peptide structure in Table 1. The peptide structure comprises the amino acid sequence of SEQ ID NOs: 12-26 identified in Table 1 as corresponding to the peptide structure.

In one or more embodiments, a kit comprises at least one agent for quantifying at least one peptide structure identified in Table 1 to carry out part or all of any one or more of the methods described herein.

In one or more embodiments, a kit comprises at least one of a glycopeptide standard, a buffer, and/or a set of peptide sequences to carry out the method of part or all of any one or more of the methods described herein. A peptide sequence of the set of peptide sequences is identified by a corresponding one of SEQ ID NOS: 12-26, defined in Table 1.

In one or more embodiments, a system comprises one or more data processors; and a non-transitory computer readable storage medium containing instructions which, when executed on the one or more data processors, cause the one or more data processors to perform part or all of any one or more of the methods described herein.

In one or more embodiments, a computer-program product tangibly embodied in a non-transitory machine-readable storage medium is provided that includes instructions configured to cause one or more data processors to perform part or all of any one or more of the methods described herein.

### BRIEF DESCRIPTION OF THE DRAWINGS

The present disclosure is described in conjunction with the appended figures:

FIG. 1 is a schematic diagram of an exemplary workflow 100 for the detection of peptide structures associated with a disease state for use in diagnosis and/or treatment in accordance with one or more embodiments.

FIG. 2A is a schematic diagram of a preparation workflow in accordance with one or more embodiments.

FIG. 2B is a schematic diagram of data acquisition in accordance with one or more embodiments.

FIG. 3 is a block diagram of an analysis system in accordance with one or more embodiments.

FIG. 4 is a block diagram of a computer system in accordance with various embodiments.

FIG. 5 is a flowchart of a process for managing a treatment for a subject diagnosed with sarcoma in accordance with one or more embodiments.

FIG. 6 is a flowchart of a process for building a model to predict treatment responsiveness for a subject diagnosed with a sarcoma disease state in accordance with one or more embodiments.

FIG. 7 is a graph describing the survival information for the 43-patient cohort in accordance with one or more embodiments.

FIG. 8 is a plot 800 of the distribution of the response scores generated for the final model described above in accordance with one or more embodiments

FIG. 9 is a plot of time versus percentage of survival in accordance with one or more embodiments.

### DETAILED DESCRIPTION

### I. Overview

The embodiments described herein recognize that glycoproteomics is an emerging field that can be used in the overall diagnosis and/or treatment of subjects with various types of diseases. Glycoproteomics aims to determine the positions, identities, and quantities of glycans and glycosylated proteins in a given sample (e.g., blood sample, cell, tissue, etc.). Protein glycosylation is one of the most common and most complex forms of post-translational protein modification, and can affect protein structure, conformation, and function. For example, glycoproteins may play crucial roles in important biological processes such as cell signaling, host-pathogen interactions, and immune response and disease. Glycoproteins may therefore be important to diagnosing different types of diseases.

Although protein glycosylation provides useful information about cancer and other diseases, analysis of protein glycosylation may be difficult as the glycan typically cannot be traced back to the protein site of origin with currently available methodologies. Glycoprotein analysis can be challenging in general due to several reasons. For example, a single glycan composition in a peptide may contain a large number of isomeric structures because of different glycosidic linkages, branching, and many monosaccharides having the same mass. Further, the presence of multiple glycans that share the same peptide sequence may cause the mass spectrometry (MS) signal to split into various glycoforms, lowering their individual abundances compared to the peptides that are not glycosylated (aglycosylated peptides).

To understand various disease conditions and to more accurately understand how a subject diagnosed with a certain disease, such as sarcoma, may respond to treatment, it may be important to perform analysis of glycoproteins and to identify not only the glycan but also the linking site (e.g., the amino acid residue of attachment) within the protein. Thus, there is a need to provide a method for site-specific glycoprotein analysis to obtain detailed information about protein glycosylation patterns which may be able to provide information about a disease state (e.g., a sarcoma disease state). This information can be used to predict whether a subject will likely respond to treatment or will likely not respond to treatment.

Sarcoma is a type of cancer (malignant tumor) that arises in connective tissue, which includes bone and soft tissue. Soft tissue sarcoma can form in muscle, fat, blood vessels, nerves, tendons, the lining of joints, or a combination thereof. While surgery is currently the most common form of treatment for most sarcomas, other types of treatment include chemotherapy, radiation therapy, proton therapy, and/or immunotherapy. One example of an immunotherapy that may be used to treat sarcoma (or a sarcoma disease state) includes one or more antibodies, such as one or more monoclonal antibodies, e.g., a combination of durvalumab and tremelimumab, such as at 1500 mg intravenous durvalumab and 75 mg intravenous tremelimumab for four cycles, followed by durvalumab alone every 4 weeks for up to 12 months.

Different patients may respond differently to different treatments. For example, some patients may have great success with one type of treatment while other patients may have limited or no success with that same treatment. Because sarcoma can be aggressive, subjects may not have the luxury of trying different types of treatments over time. It may be important to identify those subjects who are likely to respond to a given treatment to help avoid the burden associated with adverse events (e.g., events that disrupt a subject's progression-free survival) and to avoid the cost associated with treatment subjects who are not likely to respond to certain treatments. The embodiments described herein provide a way of predicting treatment response with respect to survivability (e.g., overall survival, progression-free survival) at a baseline point in time prior to administration of the treatment.

Analyzing peptide structure expression in subjects and, in particular, glycopeptide structure abundance may help predict subject response to treatment for sarcoma. A peptide structure may be defined by an aglycosylated peptide sequence (e.g., a peptide or peptide fragment of a larger parent protein) or a glycosylated peptide sequence. A glycosylated peptide sequence (also referred to as a glycopeptide structure) may be a peptide sequence having a glycan structure that is attached to a linking site (e.g., an amino acid residue) of the peptide sequence, which may occur via, for example, a particular atom of the amino acid residue). Non-limiting examples of glycosylated peptides include N-linked glycopeptides and O-linked glycopeptides.

This type of peptide structure analysis may be more conducive to accurately predicting treatment response as compared to glycomic analysis that provides little to no information about what proteins and to which amino acid residue sites various glycan structures attach. By analyzing which peptide structures are most significantly associated with survival after administration of a given treatment, and then analyzing a subject's peptide structure profile of those particular one or more peptide structures, a clearer understanding of how that subject will respond to that treatment may be achieved.

Accordingly, the embodiments described herein provide various methods and systems for analyzing proteins in subjects and, in particular, glycoproteins. In one or more embodiments, methods and systems are provided for treatment management of a subject diagnosed with a sarcoma disease state.

For example, the embodiments described herein provide methods and systems for receiving peptide structure data corresponding to a set of glycoproteins in a biological sample obtained from the subject; computing a response score that predicts a likelihood of responsiveness to a treatment using quantification data identified from the peptide structure data for a set of peptide structures; and generating a treatment response output based on the response score. The set of peptide structures includes at least one peptide structure identified from a plurality of peptide structures listed in Table 1. The plurality of peptide structures is listed in Table 1 with respect to relative significance to an overall survival for the sarcoma disease state. The treatment response output may indicate whether the subject is likely to respond to the treatment or likely to not respond to the treatment.

The description below provides exemplary implementations of the methods and systems described herein for the research and/or treatment (e.g., designing, planning, administration, etc. of a treatment) of sarcoma. Descriptions and examples of various terms, as used herein, are provided in Section II below.

### II. Exemplary Descriptions of Terms

The term “ones” means more than one.

As used herein, the term “plurality” may be 2, 3, 4, 5, 6, 7, 8, 9, 10, or more.

As used herein, the term “set of” means one or more. For example, a set of items includes one or more items.

As used herein, the phrase “at least one of,” when used with a list of items, means different combinations of one or more of the listed items may be used and only one of the items in the list may be needed. The item may be a particular object, thing, step, operation, process, or category. In other words, “at least one of” means any combination of items or number of items may be used from the list, but not all of the items in the list may be required. For example, without limitation, “at least one of item A, item B, or item C” means item A; item A and item B; item B; item A, item B, and item C; item B and item C; or item A and C. In some cases, “at least one of item A, item B, or item C” means, but is not limited to, two of item A, one of item B, and ten of item C; four of item B and seven of item C; or some other suitable combination.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

As used herein, “abundance,” may refer to a quantitative value generated using mass spectrometry. In various embodiments, the quantitative value may relate to an amount of a particular peptide structure (e.g., biomarker) present in a biological sample. In some embodiments, the amount may be in relation to other structures present in the sample (e.g., relative abundance) In some embodiments, the quantitative value may comprise an amount of an ion produced using mass spectrometry. In some embodiments, the quantitative value may be associated with an m/z value (e.g., abundance on x-axis and m/z on y-axis).

The term “amino acid,” as used herein, generally refers to any organic compound that includes an amino group (e.g., —NH₂), a carboxyl group (—COOH), and a side chain group (R) which varies based on a specific amino acid. Amino acids can be linked using peptide bonds.

The term “alkylation,” as used herein, generally refers to the transfer of an alkyl group from one molecule to another. In various embodiments, alkylation is used to react with reduced cysteines to prevent the re-formation of disulfide bonds after reduction has been performed.

The term “linking site” or “glycosylation site” as used herein generally refers to the location where a sugar molecule of a glycan or glycan structure is directly bound (e.g., covalently bound) to an amino acid of a peptide, a polypeptide, or a protein. For example, the linking site may be an amino acid residue and a glycan structure may be linked via an atom of the amino acid residue. Non-limiting examples of types of glycosylation can include N-linked glycosylation, O-linked glycosylation, C-linked glycosylation, S-linked glycosylation, and glycation.

The terms “biological sample,” “biological specimen,” or “biospecimen” as used herein, generally refers to a specimen taken by sampling so as to be representative of the source of the specimen, typically, from a subject. A biological sample can be representative of an organism as a whole, specific tissue, cell type, or category or sub-category of interest. The biological sample can include a macromolecule. The biological sample can include a small molecule. The biological sample can include a virus. The biological sample can include a cell or derivative of a cell. The biological sample can include an organelle. The biological sample can include a cell nucleus. The biological sample can include a rare cell from a population of cells. The biological sample can include any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological sample can include a constituent of a cell. The biological sample can include nucleotides (e.g., ssDNA, dsDNA, RNA), organelles, amino acids, peptides, proteins, carbohydrates, glycoproteins, or any combination thereof. The biological sample can include a matrix (e.g., a gel or polymer matrix) comprising a cell or one or more constituents from a cell (e.g., cell bead), such as DNA, RNA, organelles, proteins, or any combination thereof, from the cell. The biological sample may be obtained from a tissue of a subject. The biological sample can include a hardened cell. Such hardened cells may or may not include a cell wall or cell membrane. The biological sample can include one or more constituents of a cell but may not include other constituents of the cell. An example of such constituents may include a nucleus or an organelle. The biological sample may include a live cell. The live cell can be capable of being cultured. The sample may include at least one of a plasma, blood, or serum sample.

The term “biomarker,” as used herein, generally refers to any measurable substance taken as a sample from a subject whose presence is indicative of some phenomenon. Non-limiting examples of such phenomenon can include a disease state, a condition, or exposure to a compound or environmental condition. In various embodiments described herein, biomarkers may be used for diagnostic purposes (e.g., to diagnose a health state, a disease state). The term “biomarker” can be used interchangeably with the term “marker.”

The term “denaturation,” as used herein, generally refers to any molecule that loses quaternary structure, tertiary structure, and secondary structure which is present in their native state. Non-limiting examples include proteins or nucleic acids being exposed to an external compound or environmental condition such as acid, base, temperature, pressure, radiation, etc.

The term “denatured protein,” as used herein, generally refers to a protein that loses quaternary structure, tertiary structure, and secondary structure which is present in their native state.

The terms “digestion” or “enzymatic digestion,” as used herein, generally refer to breaking apart a polymer (e.g., cutting a polypeptide at a cut site). Proteins may be digested in preparation for mass spectrometry using trypsin digestion protocols. Proteins may be digested using other proteases in preparation for mass spectrometry if access is limited to cleavage sites.

The term “disease state” as used herein, generally refers to a condition that affects the structure or function of an organism. Non-limiting examples of causes of disease states may include pathogens, immune system dysfunctions, cell damage caused by aging, cell damage caused by other factors (e.g., trauma and cancer). Disease states can include any state of a disease whether symptomatic or asymptomatic. Disease states can include disease stages of a disease progression. Disease states can cause minor, moderate, or severe disruptions in structure or function of an organism (e.g., a subject).

The terms “glycan” or “polysaccharide” as used herein, both generally refer to a carbohydrate residue of a glycoconjugate, such as the carbohydrate portion of a glycopeptide, glycoprotein, glycolipid, or proteoglycan. Glycans can include monosaccharides.

The term “glycopeptide” or “glycopolypeptide” as used herein, generally refer to a peptide or polypeptide comprising at least one glycan residue. In various embodiments, glycopeptides comprise carbohydrate moieties (e.g., one or more glycans) covalently attached to a side chain (i.e. R group) of an amino acid residue.

The term “glycoprotein,” as used herein, generally refers to a protein having at least one glycan residue bonded thereto. In some examples, a glycoprotein is a protein with at least one oligosaccharide chain covalently bonded thereto. Examples of glycoproteins, include but are not limited to apolipoprotein C-III (APOC3), alpha-1-antichymotrypsin (AACT), afamin (AFAM), alpha-1-acid glycoprotein 1 & 2 (AGP12), apolipoprotein B-100 (APOB), apolipoprotein D (APOD), complement Cls subcomponent (CIS), calpain-3 (CAN3), clusterin (CLUS), complement component C8AChain (CO8A), alpha-2-HS-glycoprotein (FETUA), haptoglobin (HPT), immunoglobulin heavy constant gamma 1 (IgG1), immunoglobulin J chain (IgJ), plasma kallikrein (KLKB1), serum paraoxonase/arylesterase 1 (PON1), prothrombin (THRB), serotransferrin (TRFE), protein unc-13 homologA (UN13A), and zinc-alpha-2-glycoprotein (ZA2G). A glycopeptide, as used herein, refers to a fragment of a glycoprotein, unless specified otherwise to the contrary.

The term “liquid chromatography,” as used herein, generally refers to a technique used to separate a sample into parts. Liquid chromatography can be used to separate, identify, and quantify components.

The term “mass spectrometry,” as used herein, generally refers to an analytical technique used to identify molecules. In various embodiments described herein, mass spectrometry can be involved in characterization and sequencing of proteins.

The term “m/z” or “mass-to-charge ratio” as used herein, generally refers to an output value from a mass spectrometry instrument. In various embodiments, m/z can represent a relationship between the mass of a given ion and the number of elementary charges that it carries. The “m” in m/z stands for mass and the “z” stands for charge. In some embodiments, m/z can be displayed on an x-axis of a mass spectrum.

The term “peptide,” as used herein, generally refers to amino acids linked by peptide bonds. Peptides can include amino acid chains between 10 and 50 residues. Peptides can include amino acid chains shorter than 10 residues, including, oligopeptides, dipeptides, tripeptides, and tetrapeptides. Peptides can include chains longer than 50 residues and may be referred to as “polypeptides” or “proteins.”

The terms “protein” or “polypeptide” or “peptide” may be used interchangeably herein and generally refer to a molecule including at least three amino acid residues. Proteins can include polymer chains made of amino acid sequences linked together by peptide bonds. Proteins may be digested in preparation for mass spectrometry using trypsin digestion protocols. Proteins may be digested using other proteases in preparation for mass spectrometry if access is limited to cleavage sites.

The term “peptide structure,” as used herein, generally refers to peptides or a portion thereof or glycopeptides or a portion thereof. In various embodiments described herein, a peptide structure can include any molecule comprising at least two amino acids in sequence.

The term “reduction,” as used herein, generally refers to the gain of an electron by a substance. In various embodiments described herein, a sugar can directly bind to a protein, thereby, reducing the amino acid to which it binds. Such reducing reactions can occur in glycosylation. In various embodiments, reduction may be used to break disulfide bonds between two cysteines.

The term “sample,” as used herein, generally refers to a sample from a subject of interest and may include a biological sample of a subject. The sample may include a cell sample. The sample may include a cell line or cell culture sample. The sample can include one or more cells. The sample can include one or more microbes. The sample may include a nucleic acid sample or protein sample. The sample may also include a carbohydrate sample or a lipid sample. The sample may be derived from another sample. The sample may include a tissue sample, such as a biopsy, core biopsy, needle aspirate, or fine needle aspirate. The sample may include a fluid sample, such as a blood sample, urine sample, or saliva sample. The sample may include a skin sample. The sample may include a cheek swab. The sample may include at least one of a plasma, blood, or serum sample. The sample may include a cell-free or cell free sample. A cell-free sample may include extracellular polynucleotides. The sample may originate from blood, plasma, serum, urine, saliva, mucosal excretions, sputum, stool, or tears. The sample may originate from red blood cells or white blood cells. The sample may originate from feces, spinal fluid, CNS fluid, gastric fluid, amniotic fluid, cyst fluid, peritoneal fluid, marrow, bile, other body fluids, tissue obtained from a biopsy, skin, or hair.

The term “sequence,” as used herein, generally refers to a biological sequence including one-dimensional monomers that can be assembled to generate a polymer. Non-limiting examples of sequences include nucleotide sequences (e.g., ssDNA, dsDNA, and RNA), amino acid sequences (e.g., proteins, peptides, and polypeptides), and carbohydrates (e.g., compounds including Cₘ (H₂O)ₙ).

The term “subject,” as used herein, generally refers to an animal, such as a mammal (e.g., human) or avian (e.g., bird), or other organism, such as a plant. For example, the subject can include a vertebrate, a mammal, a rodent (e.g., a mouse), a primate, a simian or a human. Animals may include, but are not limited to, farm animals, sport animals, and pets. A subject can include a healthy or asymptomatic individual, an individual that has or is suspected of having a disease (e.g., cancer) or a pre-disposition to the disease, and/or an individual that is in need of therapy or suspected of needing therapy. A subject can be a patient. A subject can include a microorganism or microbe (e.g., bacteria, fungi, archaea, viruses).

As used herein, a “model” may include one or more algorithms, one or more mathematical techniques, one or more machine learning algorithms, or a combination thereof.

As used herein, “machine learning” may be the practice of using algorithms to parse data, learn from it, and then make a determination or prediction about something in the world. Machine learning uses algorithms that can learn from data without relying on rules-based programming. A machine learning algorithm may include a parametric model, a nonparametric model, a deep learning model, a neural network, a linear discriminant analysis model, a quadratic discriminant analysis model, a support vector machine, a random forest algorithm, a nearest neighbor algorithm, a combined discriminant analysis model, a k-means clustering algorithm, a supervised model, an unsupervised model, logistic regression model, a multivariable regression model, a penalized multivariable regression model, or another type of model.

As used herein, a “target glycopeptide analyte,” may refer to a peptide structure (e.g., glycosylated or aglycosylated/non-glycosylated), a fraction of a peptide structure, a sub-structure (e.g., a glycan or a glycosylation site) of a peptide structure, a product of one or more of the above listed structures and sub-structures, associated detection molecules (e.g., signal molecule, label, or tag), or an amino acid sequence that can be measured by mass spectrometry.

As used herein, a “a peptide data set,” may be used interchangeably with “peptide structure data” and can refer to any data of or relating to a peptide from a resulting mass spectrometry run. A peptide data set can comprise data obtained from a sample or biological sample using mass spectrometry. A peptide dataset can comprise data relating to a NGEP external standard, data relating to an internal standard, and data relating to a target glycopeptide analyte of a sample. A peptide data set can result from analysis originating from a single run. In some embodiments, the peptide data set can include raw abundance and mass to charge ratios for one or more peptides.

As used herein, a “non-glycosylated endogenous peptide” (“NGEP”), which may also be referred to as an aglycosylated peptide, may refer to a peptide structure that does not comprise a glycan molecule. In various embodiments, an NGEP and a target glycopeptide analyte can originate from the same subject. In various embodiments, an NGEP can be labeled with an isotope in preparation for mass spectrometry analysis. In various embodiments, an NGEP and a target glycopeptide analyte may be derived from the same protein sequence. In some embodiments, the NGEP and the target glycopeptide analyte may be derived from or include the same peptide sequence. In various embodiments, a NGEP can be labeled with an isotope in preparation for mass spectrometry analysis.

As used herein, a “a transition,” may refer to or identify a peptide structure. In some embodiments, a transition can refer to the specific pair of m/z values associated with a precursor ion and a product or fragment ion.

As used herein, “abundance,” may refer to a quantitative value generated using mass spectrometry. In various embodiments, the quantitative value may relate to the amount of a particular peptide structure. In some embodiments, the quantitative value may comprise an amount of an ion produced using mass spectrometry. In some embodiments, the quantitative value may be expressed as an m/z value. In other embodiments, the quantitative value may be expressed in atomic mass units.

As used herein, “relative abundance,” may refer to a comparison of two or more abundances. In various embodiments, the comparison may comprise comparing one peptide structure to a total number of peptide structures. In some embodiments, the comparison may comprise comparing one peptide glycoform (e.g., two identical peptides differing by one or more glycans) to a set of peptide glycoforms. In some embodiments, the comparison may comprise comparing a number of ions having a particular m/z ratio by a total number of ions detected. In various embodiments, a relative abundance can be expressed as a ratio. In other embodiments, a relative abundance can be expressed as a percentage. Relative abundance can be presented on a y-axis of a mass spectrum plot.

As used herein, an “internal standard,” may refer to something that can be contained (e.g., spiked-in) in the same sample as a target glycopeptide analyte undergoing mass spectrometry analysis. Internal standards can be used for calibration purposes. Additionally, internal standards can be used in the systems and method described herein. In some aspects, an internal standard can be selected based on similarity m/z and or retention times and can be a “surrogate” if a specific standard is too costly or unavailable. Internal standards can be heavy labeled or non-heavy labeled.

### III. Overview of Exemplary Workflow

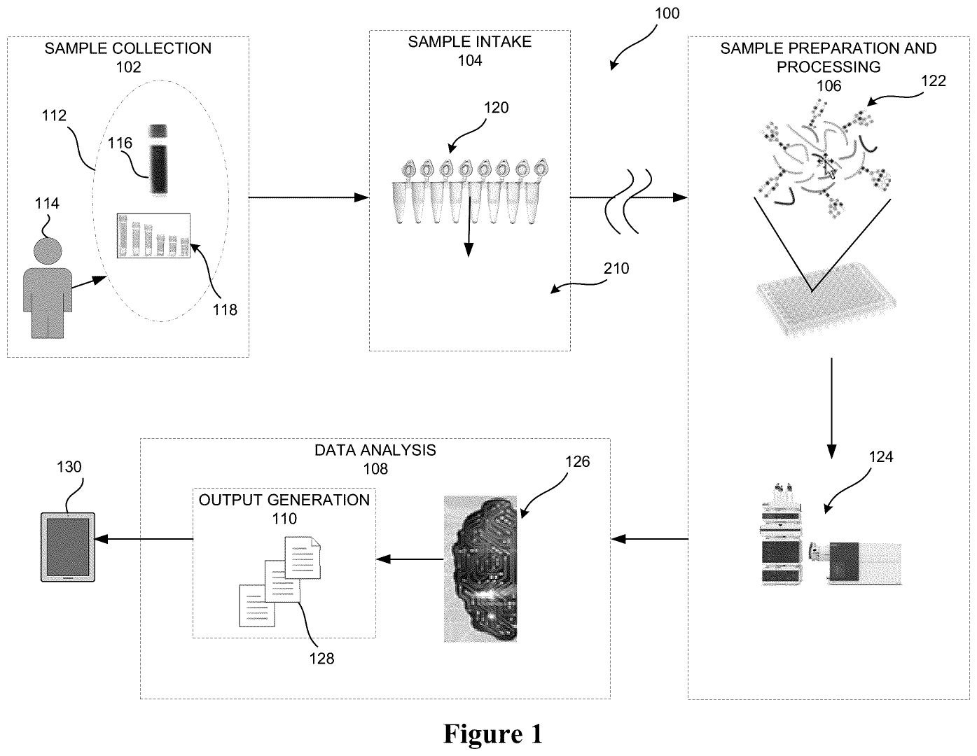

FIG. 1 is a schematic diagram of an exemplary workflow 100 for the detection of peptide structures associated with a disease state for use in diagnosis and/or treatment in accordance with one or more embodiments. Workflow 100 may include various operations including, for example, sample collection 102, sample intake 104, sample preparation and processing 106, data analysis 108, and output generation 110.

Sample collection 102 may include, for example, obtaining a biological sample 112 of one or more subjects, such as subject 114. Biological sample 112 may take the form of a specimen obtained via one or more sampling methods. Biological sample 112 may be representative of subject 114 as a whole or of a specific tissue, cell type, or other category or sub-category of interest. Biological sample 112 may be obtained in any of a number of different ways. In various embodiments, biological sample 112 includes whole blood sample 116 obtained via a blood draw. In other embodiments, biological sample 112 includes set of aliquoted samples 118 that includes, for example, a serum sample, a plasma sample, a blood cell (e.g., white blood cell (WBC), red blood cell (RBC) sample, another type of sample, or a combination thereof. Biological samples 112 may include nucleotides (e.g., ssDNA, dsDNA, RNA), organelles, amino acids, peptides, proteins, carbohydrates, glycoproteins, or any combination thereof.

In various embodiments, a single run can analyze a sample (e.g., the sample including a peptide analyte), an external standard (e.g., an NGEP of a serum sample), and an internal standard. As such, abundance values (e.g., abundance or raw abundance) for the external standard, the internal standard, and target glycopeptide analyte can be determined by mass spectrometry in the same run.

In various embodiments, external standards may be analyzed prior to analyzing samples. In various embodiments, the external standards can be run independently between the samples. In some embodiments, external standards can be analyzed after every 1, 2, 3, 4, 5, 6, 7, 8, 9, 10, 11, 12, 13, 14, 15, 16, 17, 18, 19, 20, or more experiments. In various embodiments, external standard data can be used in some or all of the normalization systems and methods described herein. In additional embodiments, blank samples may be processed to prevent column fouling.

Sample intake 104 may include one or more various operations such as, for example, aliquoting, registering, processing, storing, thawing, and/or other types of operations. In one or more embodiments, when biological sample 112 includes whole blood sample 116, sample intake 104 includes aliquoting whole blood sample 116 to form a set of aliquoted samples that can then be sub-aliquoted to form set of samples 120.

Sample preparation and processing 106 may include, for example, one or more operations to form set of peptide structures 122. In various embodiments, set of peptide structures 122 may include various fragments of unfolded proteins that have undergone digestion and may be ready for analysis.

Further, sample preparation and processing 106 may include, for example, data acquisition 124 based on set of peptide structures 122. For example, data acquisition 124 may include use of, for example, but is not limited to, a liquid chromatography/mass spectrometry (LC/MS) system.

Data analysis 108 may include, for example, peptide structure analysis 126. In some embodiments, data analysis 108 also includes output generation 110. In other embodiments, output generation 110 may be considered a separate operation from data analysis 108. Output generation 110 may include, for example, generating final output 128 based on the results of peptide structure analysis 126. Final output 128 may be used for determining research, diagnosis, and/or treatment.

In various embodiments, final output 128 is comprised of one or more outputs. Final output 128 may take various forms. For example, final output 128 may be a report that includes, for example, a diagnosis output, a treatment output (e.g., a treatment design output, a treatment plan output, or combination thereof), analyzed data (e.g., relativized and normalized) or combination thereof. In some embodiments, report can comprise a target glycopeptide analyte concentration as a function of the NGEP concentration value and the normalized abundance value. In some embodiments, final output 128 may be an alert (e.g., a visual alert, an audible alert, etc.), a notification (e.g., a visual notification, an audible notification, an email notification, etc.), an email output, or a combination thereof. In some embodiments, final output 128 may be sent to remote system 130 for processing. Remote system 130 may include, for example, a computer system, a server, a processor, a cloud computing platform, cloud storage, a laptop, a tablet, a smartphone, some other type of mobile computing device, or a combination thereof.

In other embodiments, workflow 100 may optionally exclude one or more of the operations described herein and/or may optionally include one or more other steps or operations other than those described herein (e.g., in addition to and/or instead of those described herein). For example, in one or more embodiments, final output 128 may not be sent to remote system 130 for processing. Instead, a notification or a communication (e.g., email) may be sent to remote system 130 to notify a user(s) or entity that final output 128 is available for retrieval (e.g., download). Accordingly, workflow 100 may be implemented in any of a number of different ways for use in the research, diagnosis, and/or treatment of a disease state.

### IV. Detection and Quantification of Peptide Structures

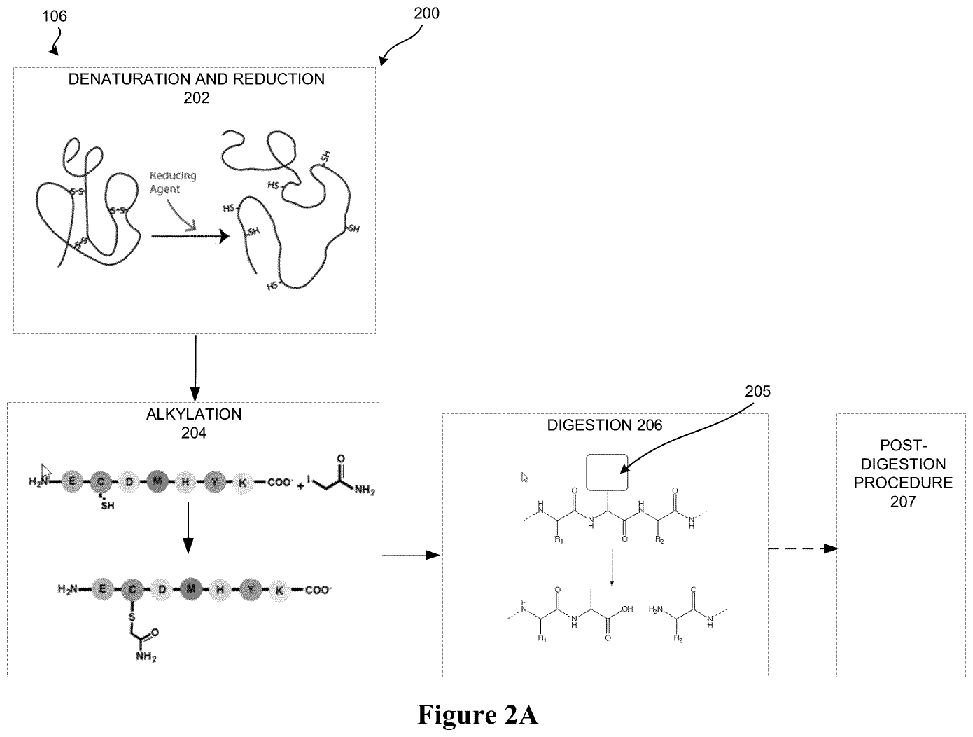

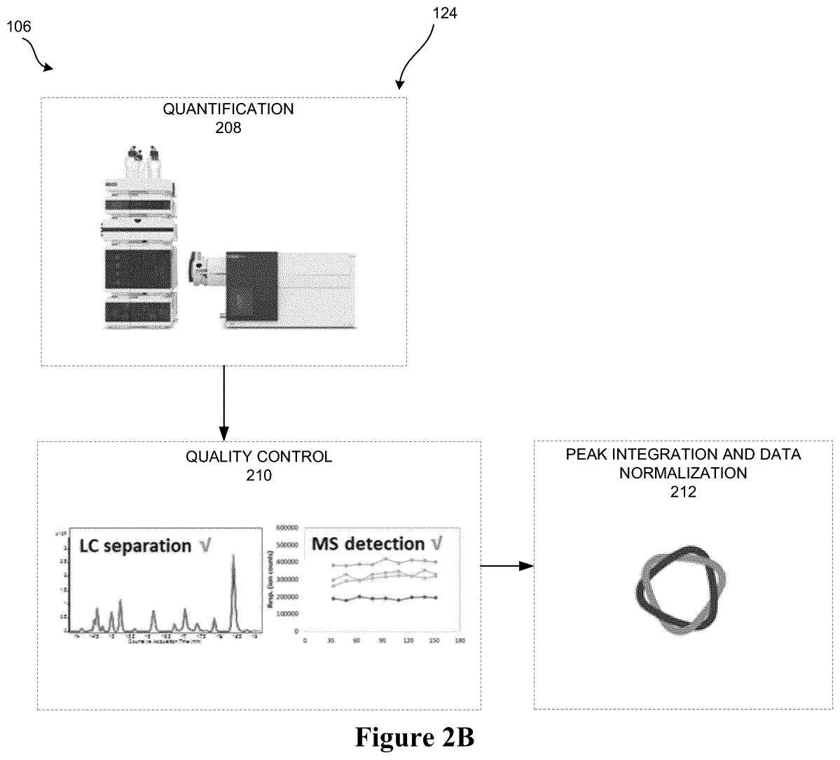

FIGS. 2A and 2B are schematic diagrams of a workflow for sample preparation and processing 106 in accordance with one or more embodiments. FIGS. 2A and 2B are described with continuing reference to FIG. 1. Sample preparation and processing 106 may include, for example, preparation workflow 200 shown in FIG. 2A and data acquisition 124 shown in FIG. 2B.

### IV.A. Sample Preparation and Processing

FIG. 2A is a schematic diagram of preparation workflow 200 in accordance with one or more embodiments. Preparation workflow 200 may be used to prepare a sample, such as a sample of set of samples 120 in FIG. 1, for analysis via data acquisition 124. For example, this analysis may be performed via mass spectrometry (e.g., LC-MS). In various embodiments, preparation workflow 200 may include denaturation and reduction 202, alkylation 204, and digestion 206.

In general, polymers, such as proteins, in their native form, can fold to include secondary, tertiary, and/or other higher order structures. Such higher order structures may functionalize proteins to complete tasks (e.g., enable enzymatic activity) in a subject. Further, such higher order structures of polymers may be maintained via various interactions between side chains of amino acids within the polymers. Such interactions can include ionic bonding, hydrophobic interactions, hydrogen bonding, and disulfide linkages between cysteine residues. However, when using analytic systems and methods, including mass spectrometry, unfolding such polymers (e.g., peptide/protein molecules) may be desired to obtain sequence information. In some embodiments, unfolding a polymer may include denaturing the polymer, which may include, for example, linearizing the polymer.

In one or more embodiments, denaturation and reduction 202 can be used to disrupt higher order structures (e.g., secondary, tertiary, quaternary, etc.) of one or more proteins (e.g., polypeptides and peptides) in a sample (e.g., one of set of samples 120 in FIG. 1). Denaturation and reduction 202 includes, for example, a denaturation procedure and a reduction procedure. In some embodiments, the denaturation procedure may be performed using, for example, thermal denaturation, where heat is used as a denaturing agent. The thermal denaturation can disrupt ionic bonding, hydrophobic interactions, and/or hydrogen bonding.

In various embodiments, the denaturation procedure may include using one or more denaturing agents, temperature (e.g., heat), or a combination thereof. In one or more embodiments, the denaturation procedure may include using one or more denaturing agents in combination with heat. These one or more denaturing agents may include, for example, but are not limited to, any number of chaotropic salts (e.g., urea, guanidine), surfactants (e.g., sodium dodecyl sulfate (SDS), beta octyl glucoside, Triton X-100), or combination thereof. In some cases, such denaturing agents may be used in combination with heat when sample preparation workflow further includes a cleanup procedure.

The resulting one or more denatured (e.g., unfolded, linearized) proteins may then undergo further processing in preparation of analysis. For example, a reduction procedure may be performed in which one or more reducing agents are applied. In various embodiments, a reducing agent can produce an alkaline pH. A reducing agent may take the form of, for example, without limitation, dithiothreitol (DTT), tris(2-carboxyethyl) phosphine (TCEP), or some other reducing agent. The reducing agent may reduce (e.g., cleave) the disulfide linkages between cysteine residues of the one or more denatured proteins to form one or more reduced proteins.

In various embodiments, the one or more reduced proteins resulting from denaturation and reduction 202 may undergo a process to prevent the reformation of disulfide linkages between, for example, the cysteine residues of the one or more reduced proteins. This process may be implemented using alkylation 204 to form one or more alkylated proteins. For example, alkylation 204 may be used to add an acetamide group to a sulfur on each cysteine residue to prevent disulfide linkages from reforming. In various embodiments, an acetamide group can be added by reacting one or more alkylating agents with a reduced protein. The one or more alkylating agents may include, for example, one or more acetamide salts. An alkylating agent may take the form of, for example, iodoacetamide (IAA), 2-chloroacetamide, some other type of acetamide salt, or some other type of alkylating agent.

In some embodiments, alkylation 204 may include a quenching procedure. The quenching procedure may be performed using one or more reducing agents (e.g., one or more of the reducing agents described above).

In various embodiments, the one or more alkylated proteins formed via alkylation 204 can then undergo digestion 206 in preparation for analysis (e.g., mass spectrometry analysis). Digestion 206 of a protein may include cleaving the protein at or around one or more cleavage sites (e.g., site 205 which may be one or more amino acid residues). For example, without limitation, an alkylated protein may be cleaved at the carboxyl side of the lysine or arginine residues. This type of cleavage may break the protein into various segments, which include one or more peptide structures (e.g., glycosylated or aglycosylated).

In various embodiments, digestion 206 is performed using one or more proteolysis catalysts. For example, an enzyme can be used in digestion 206. In some embodiments, the enzyme takes the form of trypsin. In other embodiments, one or more other types of enzymes (e.g., proteases) may be used in addition to or in place of trypsin. These one or more other enzymes include, but are not limited to, LysC, LysN, AspN, GluC, and ArgC. In some embodiments, digestion 206 may be performed using tosyl phenylalanyl chloromethyl ketone (TPCK)-treated trypsin, one or more engineered forms of trypsin, one or more other formulations of trypsin, or a combination thereof. In some embodiments, digestion 206 may be performed in multiple steps, with each involving the use of one or more digestion agents. For example, a secondary digestion, tertiary digestion, etc. may be performed. In one or more embodiments, trypsin is used to digest serum samples. In one or more embodiments, trypsin/LysC cocktails are used to digest plasma samples.

In some embodiments, digestion 206 further includes a quenching procedure. The quenching procedure may be performed by acidifying the sample (e.g., to a pH<3). In some embodiments, formic acid may be used to perform this acidification.

In various embodiments, preparation workflow 200 further includes post-digestion procedure 207. Post-digestion procedure 207 may include, for example, a cleanup procedure. The cleanup procedure may include, for example, the removal of unwanted components in the sample that results from digestion 206. For example, unwanted components may include, but are not limited to, inorganic ions, surfactants, etc. In some embodiments, post-digestion procedure 207 further includes a procedure for the addition of heavy-labeled peptide internal standards.

Although preparation workflow 200 has been described with respect to a sample created or taken from biological sample 112 that is blood-based (e.g., a whole blood sample, a plasma sample, a serum sample, etc.), sample preparation workflow 200 may be similarly implemented for other types of samples (e.g., tears, urine, tissue, interstitial fluids, sputum, etc.) to produce set of peptides structures 122.

### IV.B. Peptide Structure Identification and Quantitation

FIG. 2B is a schematic diagram of data acquisition 124 in accordance with one or more embodiments. In various embodiments, data acquisition 124 can commence following sample preparation 200 described in FIG. 2A. In various embodiments, data acquisition 124 can comprise quantification 208, quality control 210, and peak integration and normalization 212.

In various embodiments, targeted quantification 208 of peptides and glycopeptides can incorporate use of liquid chromatography-mass spectrometry LC/MS instrumentation. For example, LC-MS/MS, or tandem MS may be used. In general, LC/MS (e.g., LC-MS/MS) can combine the physical separation capabilities of liquid chromatograph (LC) with the mass analysis capabilities of mass spectrometry (MS). According to some embodiments described herein, this technique allows for the separation of digested peptides to be fed from the LC column into the MS ion source through an interface.

In various embodiments, any LC/MS device can be incorporated into the workflow described herein. In various embodiments, an instrument or instrument system suited for identification and targeted quantification 208 may include, for example, a Triple Quadrupole LC/MSTM. In various embodiments, targeted quantification 208 is performed using multiple reaction monitoring mass spectrometry (MRM-MS).

In various embodiments described herein, identification of a particular protein or peptide and an associated quantity can be assessed. In various embodiments described herein, identification of a particular glycan and an associated quantity can be assessed. In various embodiments described herein, particular glycans can be matched to a glycosylation site on a protein or peptide and the abundance values measured.

In some cases, targeted quantification 208 includes using a specific collision energy associated for the appropriate fragmentation to consistently see an abundant product ion. Glycopeptide structures may have a lower collision energy than aglycosylated peptide structures. When analyzing a sample that includes glycopeptide structures, the source voltage and gas temperature may be lowered as compared to generic proteomic analysis.

In various embodiments, quality control 210 procedures can be put in place to optimize data quality. In various embodiments, measures can be put in place allowing only errors within acceptable ranges outside of an expected value. In various embodiments, employing statistical models (e.g., using Westgard rules) can assist in quality control 210. For example, quality control 210 may include, for example, assessing the retention time and abundance of representative peptide structures (e.g., glycosylated and/or aglycosylated) and spiked-in internal standards, in either every sample, or in each quality control sample (e.g., pooled serum digest).

Peak integration and normalization 212 may be performed to process the data that has been generated and transform the data into a format for analysis. For example, peak integration and normalization 212 may include converting abundance data for various product ions that were detected for a selected peptide structure into a single quantification metric (e.g., a relative quantity, an adjusted quantity, a normalized quantity, a relative concentration, an adjusted concentration, a normalized concentration, etc.) for that peptide structure. In some embodiments, peak integration and normalization 212 may be performed using one or more of the techniques described in U.S. Patent Publication No. 2020/0372973A1 and/or US Patent Publication No. 2020/0240996A1, the disclosures of which are incorporated by reference herein in their entireties.

### V. Peptide Structure Data Analysis

### V.A. Exemplary System for Peptide Structure Data Analysis

### V.A.1. Analysis System for Peptide Structure Data Analysis and Treatment Response Prediction

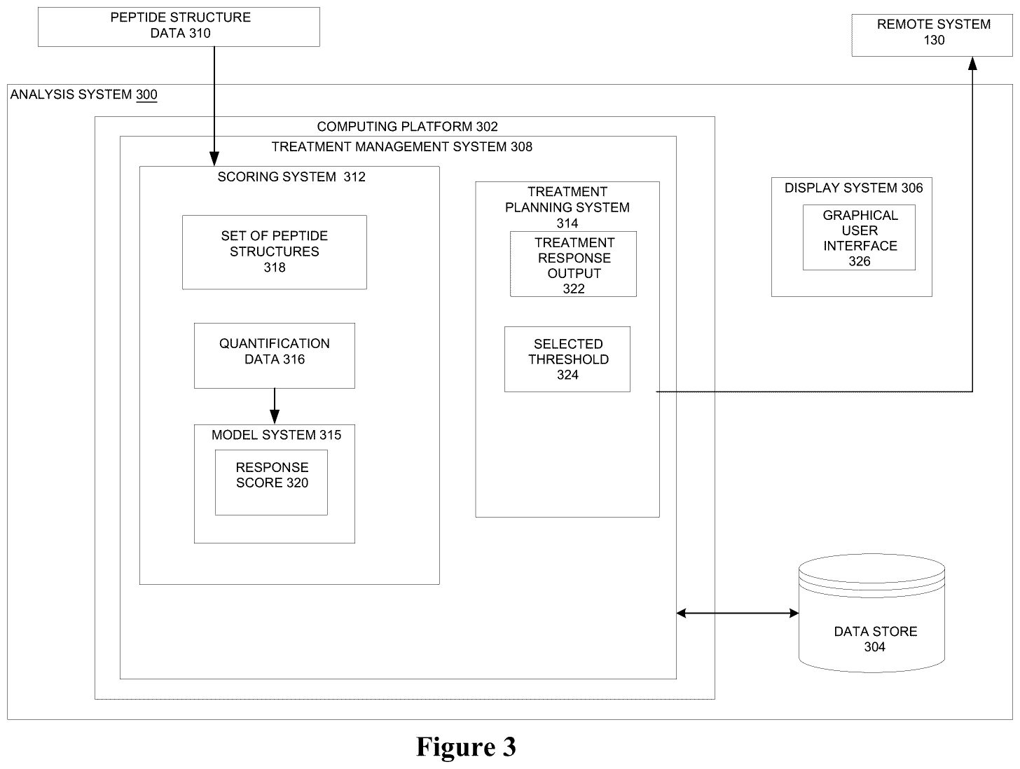

FIG. 3 is a block diagram of an analysis system 300 in accordance with one or more embodiments. Analysis system 300 can be used to both detect and analyze various peptide structures that have been associated to various disease states. Analysis system 300 is one example of an implementation for a system that may be used to perform data analysis 108 in FIG. 1. Thus, analysis system 300 is described with continuing reference to workflow 100 as described in FIGS. 1, 2A, and/or 2B.

Analysis system 300 may include computing platform 302 and data store 304. In some embodiments, analysis system 300 also includes display system 306. Computing platform 302 may take various forms. In one or more embodiments, computing platform 302 includes a single computer (or computer system) or multiple computers in communication with each other. In other examples, computing platform 302 takes the form of a cloud computing platform.

Data store 304 and display system 306 may each be in communication with computing platform 302. In some examples, data store 304, display system 306, or both may be considered part of or otherwise integrated with computing platform 302. Thus, in some examples, computing platform 302, data store 304, and display system 306 may be separate components in communication with each other, but in other examples, some combination of these components may be integrated together. Communication between these different components may be implemented using any number of wired communications links, wireless communications links, optical communications links, or a combination thereof.

Analysis system 300 includes, for example, treatment management system 308, which may be implemented using hardware, software, firmware, or a combination thereof. In one or more embodiments, treatment management system 308 is implemented using computing platform 302.

Treatment management system 308 may be used to manage the treatment of a subject diagnosed with sarcoma (i.e., a sarcoma disease state). The sarcoma may be a soft tissue sarcoma or a non-soft tissue sarcoma. The sarcoma may be of any type, including Alveolar Soft Part Sarcoma (ASPS); Angiosarcoma; Chondrosarcoma; Dermatofibrosarcoma Protuberens; Desmoid Sarcoma; Ewing's Sarcoma; Fibrosarcoma; Gastrointestinal Stromal Tumor (GIST); Non-Uterine Leiomyosarcoma; Uterine Leiomyosarcoma; Liposarcoma; Malignant Fibro Histiocytoma (MFH); Malignant Peripheral Nerve Sheath Tumor (MPNST); Osteosarcoma; Rhabdomyosarcoma; or Synovial Sarcoma. The subject may or may not be a pediatric subject or an adult subject. The subject may have been exposed to phenoxyacetic acid, such as in herbicides, and/or chlorophenols, such as in wood preservatives. The subject may have Li-Fraumeni syndrome or von Recklinghausen's disease.

Treatment management system 308 may be used to predict the subject's response to a treatment for the sarcoma disease state, determine whether to administer the treatment based on the predicted response, determine whether to modify and/or otherwise change the treatment based on the predicted response, and/or otherwise plan the treatment of the subject.

Treatment management system 308 receives peptide structure data 310 for processing. Peptide structure data 310 may have been generated using multiple reaction monitoring mass spectrometry. Peptide structure data 310 may be, for example, the peptide structure data that is output from sample preparation and processing 106 in FIGS. 1, 2A, and 2B. Accordingly, peptide structure data 310 may correspond to set of peptide structures 122 identified for biological sample 112 and may thereby correspond to biological sample 112. Further, as set of peptide structures 122 corresponds to a set of glycoproteins (e.g., each peptide structure of set of peptide structures 122 being derived from a corresponding glycoprotein), peptide structure data 310 therefore corresponds to the set of glycoproteins. In some cases, two or more peptide structures may correspond to a same glycoprotein and these two or more peptide structures may be referred to as glycoforms of that same glycoprotein.

Peptide structure data 310 can be sent as input into treatment management system 308, retrieved from data store 304 or some other type of storage (e.g., cloud storage), accessed from cloud storage, or obtained in some other manner. In some cases, peptide structure data 310 may be retrieved from data store 304 in response to (e.g., directly or indirectly based on) receiving user input entered by a user via an input device.

Treatment management system 308 may include scoring system 312, treatment planning system 314, or both. Scoring system 312 may be used to predict the response of a subject (e.g., subject 114) to a treatment. Treatment planning system 314 may be used to determine whether to and/or how to treat the subject based on the predicted response for the subject.

Scoring system 312 may include, for example, model 315 that is configured to receive peptide structure data 310 for processing. Model 315 may be implemented in any of a number of different ways. Model 315 may be a computational model system that may be implemented using any number of models, functions, equations, algorithms, and/or other mathematical techniques.

In one or more embodiments, scoring system 312 receives peptide structure data 310 for processing and inputs quantification data 316 identified from peptide structure data 310 for set of peptide structures 318 into model 315. Peptide structure data 310 may comprise a set of quantification metrics for each peptide structure of, for example, set of peptide structures 122 in FIG. 1. A quantification metric for a peptide structure may be comprised of at least one of a relative abundance, a normalized abundance, an adjusted abundance, a relative quantity, an adjusted quantity, a normalized quantity, a relative concentration, an adjusted concentration, or a normalized concentration. Accordingly, quantification data 316 may include one or more quantification metrics for each peptide structure of set of peptide structures 318.

A peptide structure of set of peptide structures 318 may be a glycosylated peptide structure, or glycopeptide structure, that is defined by a peptide sequence and a glycan structure attached to a linking site of the peptide sequence quantity. For example, the peptide structure may be a glycopeptide or a portion of a glycopeptide. Alternatively, a peptide structure of set of peptide structures 318 may be an aglycosylated peptide structure that is defined by a peptide sequence. For example, the peptide structure may be a peptide or a portion of a peptide and may be referred to as a quantification peptide.

Set of peptide structures 318 may be identified as being those most predictive or relevant to the response of a subject to a corresponding treatment(s) based on training or fitting of model 315. In one or more embodiments, set of peptide structures 318 includes at least one, at least two, at least three, at least four, at least five, at least six, at least seven, at least eight, at least nine, at least ten, at least 11, at least 12, at least 13, at least 14, at least 15, at least 16, at least 17, at least 18, at least 19, at least 20, at least 21, or all 22 of the peptide structures identified in Table 1 below in Section VI.A. The number of peptide structures selected from Table 1 for inclusion in set of peptide structures 318 may be based on, for example, a desired level of accuracy, one or more other factors, or a combination thereof.

Model 315 analyzes quantification data 316 for set of peptide structures 318 to generate response score 320 corresponding to a selected treatment for sarcoma. The selected treatment may be, for example, an immunotherapy. The immunotherapy is a combination immunotherapy that includes durvalumab and tremelimumab.

Treatment planning system 314 receives response score 320 from scoring system 312. Treatment planning system 314 uses response score 320 to generate treatment response output 322. Treatment response output 322 may be one example of an output in final output 128 described above with respect to FIG. 1. Treatment response output 322 may include, for example, an identification or categorization of the response of the subject to the treatment, at least one of an identification of a therapeutic (e.g., immunotherapy) to treat the subject, a design for the therapeutic, a treatment plan for administering the therapeutic, or a combination thereof. In various embodiments, treatment response output 322 includes a therapeutic dosage for each therapeutic to be used in treating the subject.

In one or more embodiments, treatment response output 334 identifies a response classification that indicates a predicted response for the subject to a treatment. For example, response score 320 may be used to predict whether a subject will be a likely responder to treatment or a likely non-responder to treatment.

For example, a prediction of “likely non-responder” may mean that the subject is predicted to have a disruption event within some given period of time (e.g., 3 months, 6 months, one year, two years, etc.) after treatment. A disruption event may be any event that disrupts the subject's survival (e.g., overall survival or “progression-free survival” (PFS)). For example, a disruption event may include, for example, at least one of a new tumor, an increase in the size of an existing sarcoma, death, or some other type of event. A prediction of “likely responder” may indicate that the subject is predicted to have a relatively successful response to the treatment. For example, a prediction of “likely responder” may mean that the subject is predicted to survive indefinitely or survive without a disruption event for some period of time (e.g., 3 months, 6 months, one year, two years, etc.) after treatment.

In one or more embodiments, the response score is a probability that the subject will respond to the treatment based on a set of survival criteria. The set of survival criteria may include, for example, an overall survival greater than a selected period of time (e.g., 1 month, 3 months, 6 months, one year, two years, 100 days, 200 days, 500 days, etc.). The set of survival criteria may include, for example, a progression free survival greater than a selected period of time (e.g., 1 month, 3 months, 6 months, one year, two years, 100 days, 200 days, 500 days, etc.).

In one or more embodiments, treatment planning system 314 uses selected threshold 324 to generate treatment response output 322 based on response score 320. In one or more embodiments, selected threshold 324 is determined based on the initial training/fitting of model 315. For example, treatment planning system 314 may determine that a response score 320 below selected threshold 324 indicates that the subject is a likely responder to treatment, whereas a response score 320 at or above selected threshold 324 indicates that the subject is a likely non-responder to treatment. In one or more embodiments, treatment planning system 314 may determine that a response score 320 at or below selected threshold 324 indicates that the subject is a likely responder to treatment, whereas a response score 320 above selected threshold 324 indicates that the subject is a likely non-responder to treatment.

Treatment response output 322 may include the response classification (e.g., “likely responder” (or responder) or “likely non-responder” (or non-responder)) that is predicted such that a user (e.g., a medical professional) can determine whether the corresponding treatment should be or should not be administered to a subject. For example, when treatment response output 322 indicates that a subject is predicted to be a likely responder, a medical professional may determine to proceed with administration of the treatment. In another example, when treatment response output 322 indicates that a subject is predicted to be a likely non-responder, a medical professional may determine to administer a different treatment, a higher dosage of the treatment, or change the treatment plan for the subject in some other way.

In one or more embodiments, treatment response output 322 may be sent to remote system 130 for processing in some examples. In other embodiments, treatment response output 322 may be displayed on graphical user interface 326 in display system 306 for viewing by a human operator. The human operator may use treatment response output 322 to manage the sarcoma treatment of the subject.

### V.A.2. Computer Implemented System

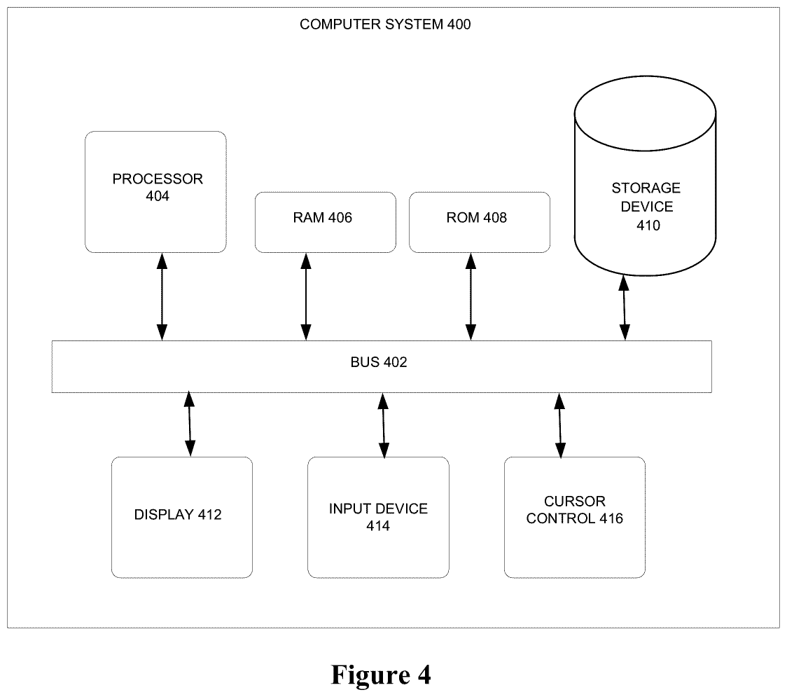

FIG. 4 is a block diagram of a computer system in accordance with various embodiments. Computer system 400 may be an example of one implementation for computing platform 302 described above in FIG. 3.

In one or more examples, computer system 400 can include a bus 402 or other communication mechanism for communicating information, and a processor 404 coupled with bus 402 for processing information. In various embodiments, computer system 400 can also include a memory, which can be a random-access memory (RAM) 406 or other dynamic storage device, coupled to bus 402 for determining instructions to be executed by processor 404. Memory also can be used for storing temporary variables or other intermediate information during execution of instructions to be executed by processor 404. In various embodiments, computer system 400 can further include a read only memory (ROM) 408 or other static storage device coupled to bus 402 for storing static information and instructions for processor 404. A storage device 410, such as a magnetic disk or optical disk, can be provided and coupled to bus 402 for storing information and instructions.

In various embodiments, computer system 400 can be coupled via bus 402 to a display 412, such as a cathode ray tube (CRT) or liquid crystal display (LCD), for displaying information to a computer user. An input device 414, including alphanumeric and other keys, can be coupled to bus 402 for communicating information and command selections to processor 404. Another type of user input device is a cursor control 416, such as a mouse, a joystick, a trackball, a gesture input device, a gaze-based input device, or cursor direction keys for communicating direction information and command selections to processor 404 and for controlling cursor movement on display 412. This input device 414 typically has two degrees of freedom in two axes, a first axis (e.g., x) and a second axis (e.g., y), that allows the device to specify positions in a plane. However, it should be understood that input devices 414 allowing for three-dimensional (e.g., x, y, and z) cursor movement are also contemplated herein.

Consistent with certain implementations of the present teachings, results can be provided by computer system 400 in response to processor 404 executing one or more sequences of one or more instructions contained in RAM 406. Such instructions can be read into RAM 406 from another computer-readable medium or computer-readable storage medium, such as storage device 410. Execution of the sequences of instructions contained in RAM 406 can cause processor 404 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, storage device, data storage device, etc.) or “computer-readable storage medium” as used herein refers to any media that participates in providing instructions to processor 404 for execution. Such a medium can take many forms, including but not limited to, non-volatile media, volatile media, and transmission media. Examples of non-volatile media can include, but are not limited to, optical, solid state, magnetic disks, such as storage device 410. Examples of volatile media can include, but are not limited to, dynamic memory, such as RAM 406. Examples of transmission media can include, but are not limited to, coaxial cables, copper wire, and fiber optics, including the wires that comprise bus 402.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 404 of computer system 400 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, optical communications connections, etc.

It should be appreciated that the methodologies described herein, flow charts, diagrams, and accompanying disclosure can be implemented using computer system 400 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 400, whereby processor 404 would execute the analyses and determinations provided by these engines, subject to instructions provided by any one of, or a combination of, the memory components RAM 406, ROM, 408, or storage device 410 and user input provided via input device 414.

V.B. Exemplary Methodologies Relating to Managing Sarcoma Treatment based on Peptide Structure Data Analysis

### V.B.1. General Methodology 20)

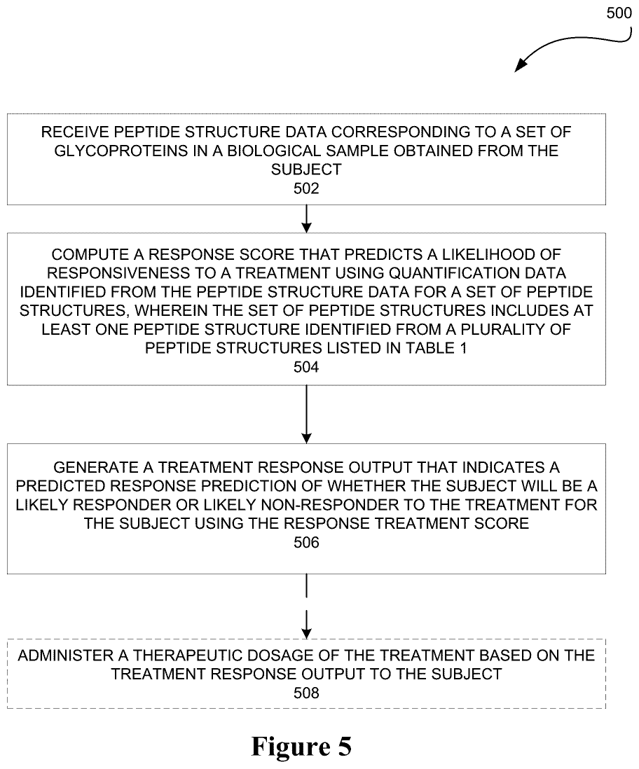

FIG. 5 is a flowchart of a process for managing a treatment for a subject diagnosed with sarcoma in accordance with one or more embodiments. Process 500 may be implemented using, for example, at least a portion of workflow 100 as described in FIGS. 1, 2A, and 2B and/or analysis system 300 as described in FIG. 3. Process 500 may be used to generate, for example, a treatment response output such as treatment response output 322 in FIG. 3 to aid in the treatment of a subject diagnosed with sarcoma (i.e., a sarcoma disease state).

Step 502 includes receiving peptide structure data corresponding to a set of glycoproteins in a biological sample obtained from the subject. The peptide structure data may be, for example, one example of an implementation of peptide structure data 310 in FIG. 3. The peptide structure data may have been generated using multiple reaction monitoring mass spectrometry. The peptide structure data may include quantification data for each peptide structure of a plurality of peptide structures. The quantification data may include, for example, one or more quantification metrics for each peptide structure of the plurality of peptide structures. A quantification metric for a peptide structure may include, for example, but is not limited to, at least one of a relative abundance, an adjusted abundance, a normalized abundance, a relative quantity, an adjusted quantity, a normalized quantity, a relative concentration, an adjusted concentration, or a normalized concentration. In this manner, the quantification data for a given peptide structure provides an indication of the abundance of the peptide structure in the biological sample.

Step 504 includes computing a response score that predicts a likelihood of responsiveness to a treatment using quantification data identified from the peptide structure data for a set of peptide structures, wherein the set of peptide structures includes at least one peptide structure identified from a plurality of peptide structures listed in Table 1. The plurality of peptide structures in Table 1 may be listed with respect to relative significance to a survival for the sarcoma disease state. In step 504, the set of peptide structures may include, for example, at least one, two, three, or some other number of peptide structures from the plurality of peptide structures identified in Table 1 below. In step 504, the set of peptide structures may include at least one glycopeptide structure defined by a peptide sequence and a glycan structure linked to a linking site of the peptide sequence, as identified in Table 1.

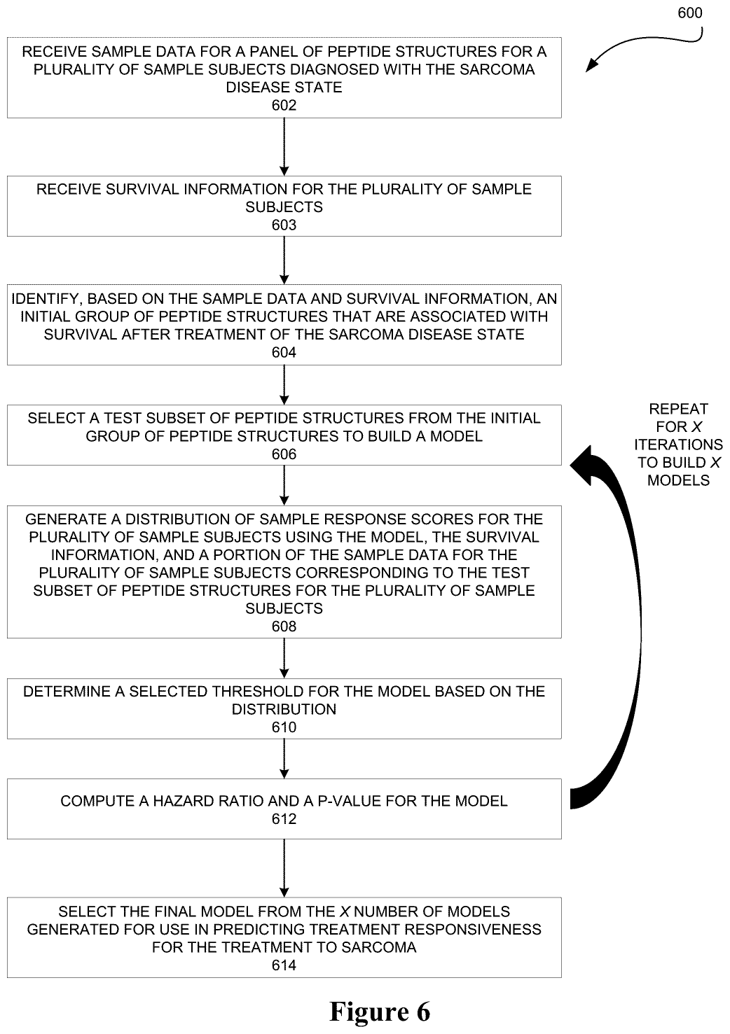

In one or more embodiments, the set of peptide structures may have been identified using sample data and survival information for a sample population (e.g., subjects diagnosed with sarcoma in which at least a portion of the subjects have been treated using the treatment being considered in process 500) and a statistical algorithm that identifies a significance of each peptide structure to survival (e.g., overall survival, progression-free survival) after treatment of sarcoma. In one or more embodiments, the identification of the set of peptide structures is performed using process 600 described below in FIG. 6.

The response score generated in step 504 may be one example of an implementation for response score 32 in FIG. 3. Step 504 may be performed by, for example, computing the response score using a model such as model 315 in FIG. 3. The model 315 may include, for example, a Cox regression model. The Cox regression model may be, for example, a Cox Proportional Hazards (PZ) regression model.

Step 506 includes generating a treatment response output that indicates a prediction of whether the subject will be a likely responder or likely non-responder to the treatment using the response score. The treatment response output may be one example of an implementation for treatment response output 322 in FIG. 3. In one or more embodiments, step 506 may be performed by determining whether the response score is above or at or above a selected threshold, such as selected threshold 324 in FIG. 3. A response score that is above (or at or above) the selected threshold may indicate that the subject is a likely non-responder to the treatment. A response score that is below (or at or below) the selected threshold may indicate that the subject is a likely responder to the treatment.

The selected threshold may have been determined from the training or fitting of the Cox regression model. For example, the selected threshold may be the cutoff response score that maximizes a concordance index (e.g., Harrell's C-index). A subject predicted to be a “likely responder” may be one who is predicted to survive indefinitely or for some period of time after treatment without a disruption event. For example, the subject may be predicted to have an overall survival or a progression-free survival that is at least some number of days, months, or years. A subject predicted to be a “likely non-responder” may be one who is predicted experience a disruption event (e.g., death, progression of the sarcoma) within some period of time after treatment. For example, the subject may be predicted to experience a disruption event within at least some number of days, months, or years.

The treatment response outcome may include, for example, a recommendation to proceed with administering the treatment if the subject is predicted to be a likely responder. Alternatively, the treatment response outcome may include, for example, a recommendation to not administer the treatment, or to modify or otherwise change a treatment or a treatment plan for the subject if the subject is predicted to be a likely non-responder. For example, a recommendation for modifying the treatment plan may include at least one of selecting a different treatment for the subject, altering (e.g., increasing/decreasing) a dosage for the treatment, or combining the treatment with at least one other treatment.

In one or more embodiments, the treatment response output includes at least one of a design for the treatment or a therapeutic dosage for the treatment. For example, in some cases when the response score indicates that the subject will respond well to the treatment, the treatment response outcome may identify the therapeutic dosage for the treatment. In this manner, a medical professional that receives the treatment response output at a remote system (e.g., phone, tablet, laptop, etc.) may be able to more quickly administer the treatment to the subject.

In one or more embodiments, process 500 may optionally include step 508. Step 508 may include administering a therapeutic dosage of the treatment based on the treatment response output to the subject. For example, the treatment may be administered (e.g., via intravenous or oral administration) based on a prediction that the subject is a likely responder to treatment.

The therapeutic dosage may be, for example, for patients over 30 kilograms, a fixed dose of 1500 mg durvalumab via intravenous (IV) infusion every 4 weeks (q4w) for up to 4 doses/cycles and 75 mg tremelimumab via IV infusion q4w for up to 4 doses/cycles, and a continuing 1500 mg durvalumab q4w starting on Week 16 for up to 9 doses/cycles.

TABLE 1
Peptide Structures associated with Sarcoma

| | | | | | Linking | Linking | |
|---|---|---|---|---|---|---|---|
| | | (Protein) | (Peptide) | Mono- | Site Pos. in | Site Pos. in | Glycan |
| PS-ID | Peptide Structure | SEQ ID | SEQ ID | isotopic | Protein | Peptide | Structure |
| NO. | (PS) NAME | NO. | NO. | mass (Da) | Sequence | Sequence | GL NO. |
| PS-1 | HEMO_64_5412 | 1 | 12 | 4877.90 | 64 | 15 | 5412 |
| PS-2 | AGP1_72MC_7603 | 2 | 13 | 6120.58 | 72 | 15 | 7603 |
| PS-3 | AGP2_72MC_7603 | 3 | 13 | 6120.58 | 72 | 15 | 7603 |
| PS-4 | A1AT_271_6503 | 4 | 14 | 4615.89 | 271 | 4 | 6503 |
| PS-5 | IC1_253_6503 | 5 | 15 | 4961.09 | 253 | 4 | 6503 |
| PS-6 | IGG1_297_3400 | 6 | 16 | 2486.98 | 180 | 5 | 3400 |
| PS-7 | HEMO_240.246_5402 | 1 | 17 | 4055.56 | 240/246 | 1/7 | 5402 |
| PS-8 | A2MG_1424_5402 | 7 | 18 | 4366.95 | 1424 | 3 | 5402 |
| PS-9 | A1AT_70_5412 | 4 | 19 | 5531.46 | 70 | 7 | 5412 |
| PS-10 | A1AT_271_5401 | 4 | 14 | 3668.56 | 271 | 4 | 5401 |
| PS-11 | A2MG_869_5402 | 7 | 20 | 5617.39 | 869 | 6 | 5402 |
| PS-12 | HPT_184 7602 | 8 | 21 | 5613.42 | 184 | 6 | 7602 |
| PS-13 | IGM_209_5411 | 9 | 22 | 4397.80 | 209 | 7 | 5411 |
| PS-14 | A2MG_869_6301 | 7 | 20 | 5285.27 | 869 | 6 | 6301 |
| PS-15 | AGP1_72_7603 | 2 | 23 | 5145.08 | 72 | 15 | 7603 |
| PS-16 | AGP2_72_7603 | 3 | 23 | 5145.08 | 72 | 15 | 7603 |
| PS-17 | A2MG_55_5402 | 7 | 24 | 4601.00 | 55 | 9 | 5402 |
| PS-18 | HRG_271_1101 | 10 | 25 | 2182.03 | 271 | 1 | 1101 |
| PS-19 | AGP1_72MC_7614 | 2 | 13 | 6557.73 | 72 | 15 | 7614 |
| PS-20 | AGP2_72MC_7614 | 3 | 13 | 6557.73 | 72 | 15 | 7614 |
| PS-21 | IGG1_297_5510 | 6 | 16 | 3160.22 | 180 | 5 | 5510 |
| PS-22 | QUANTPEP_ATL3_II | 11 | 26 | 815.48 | N/A | N/A | N/A |
| | LDGTGK | | | | | | |

Table 1 includes the Peptide Structure Identification Number (PS-ID NO.) that is a reference number for a particular peptide or glycopeptide. The Peptide Structure Name (PS-Name, e.g., HEMO_64_5412), which is a reference code for the protein name (e.g., HEMO), followed by the glycan linking site position in the protein (e.g., the number 64 that is in between two underscores and represents a sequential amino acid position in protein HEMO), and followed by the glycan structure GL number (e.g., the number 5412 that is preceded by an underscore and represents a glycan composition Hex(5)HexNAc(4)Fuc(1)NeuAc(2). The Protein Sequence ID No of Table 1 corresponds to the corresponding protein name, and Uniprot ID of Table 4. The Peptide Sequence ID No of Table 1 corresponds to the corresponding peptide sequence of Table 3. The term Linking Site Pos. within Protein Sequence is a number that refers to the sequential position of an amino acid of the corresponding protein in which a glycan is attached. For the Glycan Linking Site Pos. within Protein Sequence, the amino acid position of the peptide sequence is defined by the sequentially numbered order of amino acids based on the Uniprot ID of the corresponding protein for the peptide sequence. The term Linking Site Pos. within Peptide Sequence is a number that refers to the sequential position of an amino acid of the corresponding peptide in which a glycan is attached. For the Glycan Linking Site Pos. in peptide Sequence, the amino acid position of the peptide sequence is defined by the sequentially numbered order of amino acids for the peptide sequence. The term Glycan Structure GL No. is a number that corresponds to a symbol structure and a composition of the glycan as indicated in Table 5.

In some instances of the Peptide Structure (PS) NAME, subsequent to the prefix, there is a number noted with the notation MC that indicates that there was a miscleavage at position in the peptide sequence as noted by the number.

### V.B.2. Exemplary Methodology for Building a Model to Predict Treatment Responsiveness for a Sarcoma Treatment

FIG. 6 is a flowchart of a process for building a model to predict treatment responsiveness for a subject diagnosed with a sarcoma disease state in accordance with one or more embodiments. Process 600 may be implemented using, for example, at least a portion of workflow 100 as described in FIGS. 1, 2A, and 2B and/or analysis system 300 as described in FIG. 3. In some embodiments, process 600 may be one example of an implementation of a method for building a model such as model 315 in FIG. 3 for use in computing the response score in step 504 in FIG. 5 and the selected threshold used in step 506 in FIG. 5.

Step 602 includes receiving sample data for a panel of peptide structures for a plurality of sample subjects diagnosed with the sarcoma disease state. The plurality of sample subjects may have been treated with an immunotherapy. The immunotherapy may be, for example, a combination of durvalumab and tremelimumab.

Step 603 includes receiving survival information for the plurality of sample subjects. The survival information includes characterizations of the responses of the plurality of sample subjects to the treatment. For example, the survival information may include overall survival information, progression-free survival information, or both.

Step 604 includes identifying, based on the sample data and survival information, an initial group of peptide structures that are associated with survival after treatment of the sarcoma disease state. For example, step 604 may include performing a Cox regression analysis for each different peptide structure of the collection of peptide structures. The Cox regression analysis may be performed using a Cox PH regression model that is adjusted for age and sex with survival serving as a primary independent variable. This survival variable may be, for example, overall survival, which is the time to death relative to a baseline point in time. The baseline point in time may be a time at which the sample of the subject is collected just prior to administration of the treatment. In some cases, whether a sample subject has died may not be known and the overall survival for that subject is the time to last contact with or last clinical assessment of the sample subject.

In one or more embodiments, step 604 includes identifying a selected number, N, of the most significant peptide structures with respect to survival. For example, the peptide structures having the Nlowest Cox PH p-values may be identified as the initial group of peptide structures. The selected number N may be, for example, 5, 7, 8, 10, 12 15, 20, 25, or some other number.

Step 606 includes selecting a test subset of peptide structures from the initial group of peptide structures to build a model. For example, for a first iteration, the test subset of peptide structures may be the most significant peptide structure of the initial group of peptide structures. The most significant peptide structure may be, for example, the peptide structure with the lowest p-value.

Step 608 includes generating a distribution of sample response scores for the plurality of sample subjects using the model and a portion of the sample data for the plurality of sample subjects that includes quantification data for the test subset of peptide structures and survival information for the plurality of sample subjects. This distribution may be generated using, for example, the Cox PH regression model. In one or more embodiments, this distribution may be generated using a leave one out cross-validated (LOOCV) Cox PH regression model.

With the LOOCV Cox PH regression model, a sample response score is generated for each sample subject of the plurality of sample subjects using a model fitted to the data corresponding to the other sample subjects of the plurality of sample subjects. For example, for a given sample subject, S, a multivariable Cox PH regression model is fit based on the quantification data and survival information for the remaining (or held out) sample subjects of the plurality of sample subjects (i.e., excluding sample subject S). This multivariable Cox PH regression model is then used to generate the response score for the sample subject S. This process is repeated for each sample subject of the plurality of sample subjects to build out a distribution of sample response scores.

Step 610 includes determining a selected threshold for the model based on the distribution. The selected threshold may be, for example, the response score (or cutoff response score) that maximizes a concordance index. The concordance index may be, for example, Harrell's C-index. The selected threshold enables dichotomization between likely responders and likely non-responders. For example, a response score above (or at or above) the selected threshold may indicate a likely non-responder, whereas a response score below (or at or below) the selected threshold may indicate a likely responder.

Step 612 includes computing a hazard ratio and a p-value for the model.

Steps 606-612 are repeated up to a selected number, X, of times. For example, without limitation, the selected number, X, may be a ceiling that is set to the number of samples (sample subjects) divided by 5. Setting this ceiling may help avoid overfitting of the model. For each iteration i, the i^{th} most significant marker(s) based on the p-values generated in step 604 may be used to build the model. Thus, steps 606-612 may be iterated to build a plurality of models using different subsets of the initial group of peptide structures.

After steps 606-610 have been iterated X number of times to generate X number of models, step 614 is performed. Step 614 includes selecting a model from the X number of models generated as a final model for use in predicting treatment responsiveness for the treatment to sarcoma. The final model may be selected based on the hazard ratios, the p-values, or both for the models. In one or more embodiments, the final model that is generated uses peptide structures PS-1 and PS-2 as identified in Table 1. In other embodiments, the final model uses peptide structures PS-1 through PS-3 as identified in Table 1. In still other embodiments, the final model uses peptide structures PS-1 through PS-4 as identified in Table 1. In some embodiments, the final model may be built using peptide structures PS-1, PS-2, and PS-4.

### VI. Peptide Structure and Product Ion Compositions, Kits and Reagents

Aspects of the disclosure include compositions comprising one or more of the peptide structures listed in Table 1. In some embodiments, a composition comprises a plurality of the peptide structures listed in Table 1. In some embodiments, a composition comprises 1, 2, 3, 4, 5, 6, 7, 8, 9, 10, 11, 12, 13, 14, 15, 16, 17, 18, or 19 of the peptide structures listed in Table 1. In some embodiments, a composition comprises a peptide structure having an amino acid sequence with at least 80% sequence identity, such as, for example, at least 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NOs: 12-26, listed in Table 1.

Aspects of the disclosure include compositions comprising one or more precursor ions having a defined charge and/or defined mass-to-charge (m/z) ratio, as listed in Table 2. Aspects of the disclosure include compositions comprising one or more product ions having a defined mass-to-charge (m/z) ratio, which product ions are produced by converting a peptide structure described herein (e.g., a peptide structure listed in Table 1) into a gas phase ion in a mass spectrometry system. Conversion of the peptide structure into a gas phase ion can take place using any of a variety of techniques, including, but not limited to, matrix assisted laser desorption ionization (MALDI); electron ionization (EI); electrospray ionization (ESI); atmospheric pressure chemical ionization (APCI); and/or atmospheric pressure photo ionization (APPI).

Aspects of the disclosure include compositions comprising one or more product ions produced from one or more of the peptide structures described herein (e.g., a peptide structure listed in Table 1). In some embodiments, a composition comprises a set of the product ions listed in Table 2, having an m/z ratio selected from the list provided for each peptide structure in Table 1 or Table 2.

In some embodiments, a composition comprises at least one of peptide structures PS-1 to PS-22 identified in Table 1.

In some embodiments, a composition comprises a peptide structure or a product ion. The peptide structure or product ion comprises an amino acid sequence having at least 90% sequence identity to any one of SEQ ID NOS: 12-26, as identified in Table 3, corresponding to peptide structures PS-1 to PS-22 in Table 1.

In some embodiments, the product ion is selected as one from a group consisting of product ions identified in Table 2, including product ions falling within an identified m/z range of the m/z ratio identified in Table 2 and characterized as having a precursor ion having an m/z ratio within an identified m/z range of the m/z ratio identified in Table 2. A first range for the product ion m/z ratio may be ±0.5. A second range for the production m/z ratio may be ±0.8. A third range for the product ion m/z ratio may be ±1.0. A first range for the precursor ion m/z ratio may be ±1.0; a second range for the precursor ion m/z ratio may be (±1.5). Thus, a composition may include a product ion having an m/z ratio that falls within at least one of the first range (±0.5), the second range (±0.8), or the third range (±1.0) of the product ion m/z ratio identified in Table 2, and characterized as having a precursor ion having an m/z ratio that falls within at least one of first range (±0.5), a second range (±1.0), or a third range (±1.0 of the precursor ion m/z ratio identified in Table 2.

Table 2 shows various parameters associated with the identification of the peptide and glycopeptides using LC and MRM-MS. The retention time (RT) represents the amount of time in minutes for the peptide to elute from the chromatography column. The collision energy represents the energy applied to the peptide for creating fragments (i.e., product ions) such as, for example, in the 2nd quadrupole of the triple quadrupole MS. The first precursor m/z represents a ratio value associated with an ionized form having a precursor charge for the peptide or glycopeptide. The precursor ion is associated with a first product ion having a m/z ratio that was formed from a collision and the second precursor ion is associated with a second product ion having a m/z ratio that was formed from a collision.

TABLE 2
Mass Spectrometry-Related Characteristics for the Peptide Structures
associated with Sarcoma

| | | | | | 1st | 1st | 2nd | 2nd |
|---|---|---|---|---|---|---|---|---|
| PS-ID | RT | Collision | | Precursor | Product | Product | Product | Product |
| NO. | (min) | Energy | Precursor m/z | Charge | m/z | Charge | m/z | Charge |
| PS-1 | 40.5 | 35 | 1221 | 4 | 204.1 | 1 | 1366.1 | 2 |
| PS-2 | 40.9 | 23 | 1531.9/1225.7 | 4 or 3 | 366.1 | 1 | 1550.3 | 2 |
| PS-3 | 40.9 | 23 | 1531.9/1225.7 | 4 or 3 | 366.1 | 1 | 1550.3 | 2 |
| PS-4 | 38.3 | 30 | 1155.5/1540.3 | 3 or 4 | 274.1 | 1 | 366.1 | 1 |
| PS-5 | 35.8 | 35 | 1241.8 | 4 | 204.1 | 1 | 1152.6 | 2 |
| PS-6 | 8 | 35 | 1245 | 2 | 204.1 | 1 | N/A | N/A |
| PS-7 | 7.3 | 30 | 1015.2 | 4 | 366.1 | 1 | 685.6 | 3 |
| PS-8 | 44 | 30 or 22 | 1457.3/1093.2 | 3 or 4 | 366.1 | 1 | 1183.6 | 2 |
| PS-9 | 47 | 27 | 1107.7/1384.4 | 5 or 4 | 366.1 | 1 | 366.1 | 1 |
| PS-10 | 37.5 | 30 | 1224.5 | 3 | 366.1 | 1 | 980 | 2 |
| PS-11 | 35.9 | 25 | 1124.9 | 5 | 366.1 | 1 | 1206.2 | 3 |
| PS-12 | 33.2 | 20 | 1124 | 5 | 366.1 | 1 | N/A | N/A |
| PS-13 | 25.3 | 30 | 1467 | 3 | 366.1 | 1 | N/A | N/A |
| PS-14 | 35.2 | 20 | 1058.5 | 5 | 366.1 | 1 | 1206.6 | 3 |
| PS-15 | 37.5 | 25 | 1287.8 | 4 | 366.1 | 1 | 1062.5 | 2 |
| PS-16 | 37.5 | 25 | 1287.8 | 4 | 366.1 | 1 | 1062.5 | 2 |
| PS-17 | 42.1 | 25 | 1151.7 | 4 | 366.1 | 1 | 1300.7 | 2 |
| PS-18 | 6.5 | 10 | 546.8 | 4 | 274.1 | 1 | N/A | N/A |
| PS-19 | 41.2 | 27 | 1313.1 | 5 | 366.1 | 1 | 1550.3 | 2 |
| PS-20 | 41.2 | 27 | 1313.1 | 5 | 366.1 | 1 | 1550.3 | 2 |
| PS-21 | 8 | 20 | 1054.7 | 3 | 366.1 | 1 | N/A | N/A |
| PS-22 | 13.8 | 10 | 408.7 | 2 | 590.3 | 1 | 477.2 | 1 |

Table 3 defines the peptide sequences for SEQ ID NOS: 12-26 from Table 1. Table 3 further identifies a corresponding protein SEQ ID NO. for each peptide sequence.

TABLE 3
Peptide SEQ ID NOS

| | | Corresponding |
|---|---|---|
| SEQ ID | | Protein |
| NO: | Peptide Sequence | SEQ ID NO: |
| 12 | CSDGWSFDATTLDDNGTMLFFK | 1 |
| 13 | SVQEIQATFFYFTPNKTEDTIFLR | 2, 3 |
| 14 | YLGNATAIFFLPDEGK | 4 |
| 15 | VLSNNSDANLELINTWVAK | 5 |
| 16 | EEQYNSTYR | 6 |
| 17 | NGTGHGNSTHHGPEYMR | 1 |
| 18 | VSNQTLSLFFTVLQDVPVR | 7 |
| 19 | QLAHQSNSTNIFFSPVSIATAFAMLSLGTK | 4 |
| 20 | SLGNVNFTVSAEALESQELCGTEVPSVPEHGR | 7 |
| 21 | MVSHHNLTTGATLINEQWLLTTAK | 8 |
| 22 | GLTFQQNASSMCVPDQDTAIR | 9 |
| 23 | SVQEIQATFFYFTPNK | 2, 3 |
| 24 | GCVLLSYLNETVTVSASLESVR | 7 |
| 25 | SSTTKPPFKPHGSR | 10 |
| 26 | IILDGTGK | 11 |

Table 4 identifies the proteins of SEQ ID NOS: 1-11 from Table 1. Table 4 identifies a corresponding protein abbreviation and protein name for each of protein SEQ ID NOS: 1-11. Further, Table 4 identifies a corresponding Uniprot ID for each of protein SEQ ID NOS: 1-11.

TABLE 4
Protein SEQ ID NOS

| Prot | | | | |
|---|---|---|---|---|
| SEQ ID | Protein | | Uniprot | |
| NO. | Abbrev. | Protein Name | ID | Protein Sequence |
| 1 | HEMO | Hemopexin | P02790 | MARVLGAPVALGLWSLCWSLAIATPLPPTSAHGNVAEGET |
| | | | | KPDPDVTERCSDGWSFDATTLDDNGTMLFFKGEFVWKSH |
| | | | | KWDRELISERWKNFPSPVDAAFRQGHNSVFLIKGDKVWV |
| | | | | YPPEKKEKGYPKLLQDEFPGIPSPLDAAVECHRGECQAEG |
| | | | | VLFFQGDREWFWDLATGTMKERSWPAVGNCSSALRWLG |
| | | | | RYYCFQGNQFLRFDPVRGEVPPRYPRDVRDYFMPCPGRG |
| | | | | HGHRNGTGHGNSTHHGPEYMRCSPHLVLSALTSDNHGAT |
| | | | | YAFSGTHYWRLDTSRDGWHSWPIAHQWPQGPSAVDAAFS |
| | | | | WEEKLYLVQGTQVYVFLTKGGYTLVSGYPKRLEKEVGTP |
| | | | | HGIILDSVDAAFICPGSSRLHIMAGRRLWWLDLKSGAQAT |
| | | | | WTELPWPHEKVDGALCMEKSLGPNSCSANGPGLYLIHGP |
| | | | | NLYCYSDVEKLNAAKALPQPQNVTSLLGCTH |
| 2 | AGP1 | Alpha-1-acid | P02763 | MALSWVLTVLSLLPLLEAQIPLCANLVPVPITNATLDRITG |
| | | glycoprotein 1 | | KWFYIASAFRNEEYNKSVQEIQATFFYFTPNKTEDTIFLRE |
| | | | | YQTRQDQCIYNTTYLNVQRENGTISRYVGGQEHFAHLLIL |
| | | | | RDTKTYMLAFDVNDEKNWGLSVYADKPETTKEQLGEFYE |
| | | | | ALDCLRIPKSDVVYTDWKKDKCEPLEKQHEKERKQEEGES |
| 3 | AGP2 | Alpha-1-acid | P19652 | MALSWVLTVLSLLPLLEAQIPLCANLVPVPITNATLDRITG |
| | | glycoprotein 2 | | KWFYIASAFRNEEYNKSVQEIQATFFYFTPNKTEDTIFLRE |
| | | | | YQTRQNQCFYNSSYLNVQRENGTVSRYEGGREHVAHLLF |
| | | | | LRDTKTLMFGSYLDDEKNWGLSFYADKPETTKEQLGEFY |
| | | | | EALDCLCIPRSDVMYTDWKKDKCEPLEKQHEKERKQEEG |
| | | | | ES |
| 4 | A1AT | Alpha-1- | P01009 | MPSSVSWGILLLAGLCCLVPVSLAEDPQGDAAQKTDTSHH |
| | | antitrypsin | | DQDHPTFNKITPNLAEFAFSLYRQLAHQSNSTNIFFSPVSIA |
| | | | | TAFAMLSLGTKADTHDEILEGLNFNLTEIPEAQIHEGFQELL |
| | | | | RTLNQPDSQLQLTTGNGLFLSEGLKLVDKFLEDVKKLYHS |
| | | | | EAFTVNFGDTEEAKKQINDYVEKGTQGKIVDLVKELDRDT |
| | | | | VFALVNYIFFKGKWERPFEVKDTEEEDFHVDQVTTVKVP |
| | | | | MMKRLGMFNIQHCKKLSSWVLLMKYLGNATAIFFLPDEG |
| | | | | KLQHLENELTHDIITKFLENEDRRSASLHLPKLSITGTYDLK |
| | | | | SVLGQLGITKVFSNGADLSGVTEEAPLKLSKAVHKAVLTID |
| | | | | EKGTEAAGAMFLEAIPMSIPPEVKFNKPFVFLMIEQNTKSP |
| | | | | LFMGKVVNPTQK |
| 5 | IC1 | Plasma | P05155 | MASRLTLLTLLLLLLAGDRASSNPNATSSSSQDPESLQDRG |
| | | protease C1 | | EGKVATTVISKMLFVEPILEVSSLPTTNSTTNSATKITANTT |
| | | inhibitor | | DEPTTQPTTEPTTQPTIQPTQPTTQLPTDSPTQPTTGSFCPGP |
| | | | | VTLCSDLESHSTEAVLGDALVDFSLKLYHAFSAMKKVETN |
| | | | | MAFSPFSIASLLTQVLLGAGENTKTNLESILSYPKDFTCVH |
| | | | | QALKGFTTKGVTSVSQIFHSPDLAIRDTFVNASRTLYSSSPR |
| | | | | VLSNNSDANLELINTWVAKNTNNKISRLLDSLPSDTRLVLL |
| | | | | NAIYLSAKWKTTFDPKKTRMEPFHFKNSVIKVPMMNSKK |
| | | | | YPVAHFIDQTLKAKVGQLQLSHNLSLVILVPQNLKHRLED |
| | | | | MEQALSPSVFKAIMEKLEMSKFQPTLLTLPRIKVTTSQDML |
| | | | | SIMEKLEFFDFSYDLNLCGLTEDPDLQVSAMQHQTVLELT |
| | | | | ETGVEAAAASAISVARTLLVFEVQQPFLFVLWDQQHKFPV |
| | | | | FMGRVYDPRA |
| 6 | IGG1 | Immunoglobulin | P01857 | ASTKGPSVFPLAPSSKSTSGGTAALGCLVKDYFPEPVTVSW |
| | | heavy | | NSGALTSGVHTFPAVLQSSGLYSLSSVVTVPSSSLGTQTYIC |
| | | constant | | NVNHKPSNTKVDKKVEPKSCDKTHTCPPCPAPELLGGPSV |
| | | gamma 1 | | FLFPPKPKDTLMISRTPEVTCVVVDVSHEDPEVKFNWYVD |
| | | | | GVEVHNAKTKPREEQYNSTYRVVSVLTVLHQDWLNGKE |
| | | | | YKCKVSNKALPAPIEKTISKAKGQPREPQVYTLPPSRDELT |
| | | | | KNQVSLTCLVKGFYPSDIAVEWESNGQPENNYKTTPPVLD |
| | | | | SDGSFFLYSKLTVDKSRWQQGNVFSCSVMHEALHNHYTQ |
| | | | | KSLSLSPGK |
| 7 | A2MG | Alpha-2- | P01023 | MGKNKLLHPSLVLLLLVLLPTDASVSGKPQYMVLVPSLLH |
| | | macroglobulin | | TETTEKGCVLLSYLNETVTVSASLESVRGNRSLFTDLEAEN |
| | | | | DVLHCVAFAVPKSSSNEEVMFLTVQVKGPTQEFKKRTTV |
| | | | | MVKNEDSLVFVQTDKSIYKPGQTVKFRVVSMDENFHPLN |
| | | | | ELIPLVYIQDPKGNRIAQWQSFQLEGGLKQFSFPLSSEPFQG |
| | | | | SYKVVVQKKSGGRTEHPFTVEEFVLPKFEVQVTVPKIITILE |
| | | | | EEMNVSVCGLYTYGKPVPGHVTVSICRKYSDASDCHGEDS |
| | | | | QAFCEKFSGQLNSHGCFYQQVKTKVFQLKRKEYEMKLHT |
| | | | | EAQIQEEGTVVELTGRQSSEITRTITKLSFVKVDSHFRQGIP |
| | | | | FFGQVRLVDGKGVPIPNKVIFIRGNEANYYSNATTDEHGL |
| | | | | VQFSINTTNVMGTSLTVRVNYKDRSPCYGYQWVSEEHEE |
| | | | | AHHTAYLVFSPSKSFVHLEPMSHELPCGHTQTVQAHYILN |
| | | | | GGTLLGLKKLSFYYLIMAKGGIVRTGTHGLLVKQEDMKG |
| | | | | HFSISIPVKSDIAPVARLLIYAVLPTGDVIGDSAKYDVENCL |
| | | | | ANKVDLSFSPSQSLPASHAHLRVTAAPQSVCALRAVDQSV |
| | | | | LLMKPDAELSASSVYNLLPEKDLTGFPGPLNDQDNEDCIN |
| | | | | RHNVYINGITYTPVSSTNEKDMYSFLEDMGLKAFTNSKIRK |
| | | | | PKMCPQLQQYEMHGPEGLRVGFYESDVMGRGHARLVHV |
| | | | | EEPHTETVRKYFPETWIWDLVVVNSAGVAEVGVTVPDTIT |
| | | | | EWKAGAFCLSEDAGLGISSTASLRAFQPFFVELTMPYSVIR |
| | | | | GEAFTLKATVLNYLPKCIRVSVQLEASPAFLAVPVEKEQAP |
| | | | | HCICANGRQTVSWAVTPKSLGNVNFTVSAEALESQELCGT |
| | | | | EVPSVPEHGRKDTVIKPLLVEPEGLEKETTFNSLLCPSGGE |
| | | | | VSEELSLKLPPNVVEESARASVSVLGDILGSAMQNTQNLL |
| | | | | QMPYGCGEQNMVLFAPNIYVLDYLNETQQLTPEIKSKAIG |
| | | | | YLNTGYQRQLNYKHYDGSYSTFGERYGRNQGNTWLTAF |
| | | | | VLKTFAQARAYIFIDEAHITQALIWLSQRQKDNGCFRSSGS |
| | | | | LLNNAIKGGVEDEVTLSAYITIALLEIPLTVTHPVVRNALFC |
| | | | | LESAWKTAQEGDHGSHVYTKALLAYAFALAGNQDKRKE |
| | | | | VLKSLNEEAVKKDNSVHWERPQKPKAPVGHFYEPQAPSA |
| | | | | EVEMTSYVLLAYLTAQPAPTSEDLTSATNIVKWITKQQNA |
| | | | | QGGFSSTQDTVVALHALSKYGAATFTRTGKAAQVTIQSSG |
| | | | | TFSSKFQVDNNNRLLLQQVSLPELPGEYSMKVTGEGCVYL |
| | | | | QTSLKYNILPEKEEFPFALGVQTLPQTCDEPKAHTSFQISLS |
| | | | | VSYTGSRSASNMAIVDVKMVSGFIPLKPTVKMLERSNHVS |
| | | | | RTEVSSNHVLIYLDKVSNQTLSLFFTVLQDVPVRDLKPAIV |
| | | | | KVYDYYETDEFAIAEYNAPCSKDLGNA |
| 8 | HPT | Haptoglobin | P00738 | MSALGAVIALLLWGQLFAVDSGNDVTDIADDGCPKPPEIA |
| | | | | HGYVEHSVRYQCKNYYKLRTEGDGVYTLNDKKQWINKA |
| | | | | VGDKLPECEADDGCPKPPEIAHGYVEHSVRYQCKNYYKL |
| | | | | RTEGDGVYTLNNEKQWINKAVGDKLPECEAVCGKPKNPA |
| | | | | NPVQRILGGHLDAKGSFPWQAKMVSHHNLTTGATLINEQ |
| | | | | WLLTTAKNLFLNHSENATAKDIAPTLTLYVGKKQLVEIEK |
| | | | | VVLHPNYSQVDIGLIKLKQKVSVNERVMPICLPSKDYAEV |
| | | | | GRVGYVSGWGRNANFKFTDHLKYVMLPVADQDQCIRHY |
| | | | | EGSTVPEKKTPKSPVGVQPILNEHTFCAGMSKYQEDTCYG |
| | | | | DAGSAFAVHDLEEDTWYATGILSFDKSCAVAEYGVYVKV |
| | | | | TSIQDWVQKTIAEN |
| 9 | IGM | Immunoglobulin | P01871 | GSASAPTLFPLVSCENSPSDTSSVAVGCLAQDFLPDSITFSW |
| | | heavy | | KYKNNSDISSTRGFPSVLRGGKYAATSQVLLPSKDVMQGT |
| | | constant mu | | DEHVVCKVQHPNGNKEKNVPLPVIAELPPKVSVFVPPRDG |
| | | | | FFGNPRKSKLICQATGFSPRQIQVSWLREGKQVGSGVTTD |
| | | | | QVQAEAKESGPTTYKVTSTLTIKESDWLGQSMFTCRVDHR |
| | | | | GLTFQQNASSMCVPDQDTAIRVFAIPPSFASIFLTKSTKLTC |
| | | | | LVTDLTTYDSVTISWTRQNGEAVKTHTNISESHPNATFSAV |
| | | | | GEASICEDDWNSGERFTCTVTHTDLPSPLKQTISRPKGVAL |
| | | | | HRPDVYLLPPAREQLNLRESATITCLVTGFSPADVFVQWM |
| | | | | QRGQPLSPEKYVTSAPMPEPQAPGRYFAHSILTVSEEEWNT |
| | | | | GETYTCVVAHEALPNRVTERTVDKSTGKPTLYNVSLVMS |
| | | | | DTAGTCY |
| 10 | HRG | Histidine-rich | P04196 | MKALIAALLLITLQYSCAVSPTDCSAVEPEAEKALDLINKR |
| | | Glycoprotein | | RRDGYLFQLLRIADAHLDRVENTTVYYLVLDVQESDCSVL |
| | | | | SRKYWNDCEPPDSRRPSEIVIGQCKVIATRHSHESQDLRVI |
| | | | | DFNCTTSSVSSALANTKDSPVLIDFFEDTERYRKQANKALE |
| | | | | KYKEENDDFASFRVDRIERVARVRGGEGTGYFVDFSVRNC |
| | | | | PRHHFPRHPNVFGFCRADLFYDVEALDLESPKNLVINCEVF |
| | | | | DPQEHENINGVPPHLGHPFHWGGHERSSTTKPPFKPHGSR |
| | | | | DHHHPHKPHEHGPPPPPDERDHSHGPPLPQGPPPLLPMSCS |
| | | | | SCQHATFGTNGAQRHSHNNNSSDLHPHKHHSHEQHPHGH |
| | | | | HPHAHHPHEHDTHRQHPHGHHPHGHHPHGHHPHGHHPH |
| | | | | GHHPHCHDFQDYGPCDPPPHNQGHCCHGHGPPPGHLRRR |
| | | | | GPGKGPRPFHCRQIGSVYRLPPLRKGEVLPLPEANFPSFPLP |
| | | | | HHKHPLKPDNQPFPQSVSESCPGKFKSGFPQVSMFFTHTFP |
| | | | | K |
| 11 | ATL3 | ADAMTS- | P82987 | MASWTSPWWVLIGMVFMHSPLPQTTAEKSPGAYFLPEFAL |
| | | likeProtein3 | | SPQGSFLEDTTGEQFLTYRYDDQTSRNTRSDEDKDGNWD |
| | | | | AWGDWSDCSRTCGGGASYSLRRCLTGRNCEGQNIRYKTC |
| | | | | SNHDCPPDAEDFRAQQCSAYNDVQYQGHYYEWLPRYND |
| | | | | PAAPCALKCHAQGQNLVVELAPKVLDGTRCNTDSLDMCI |
| | | | | SGICQAVGCDRQLGSNAKEDNCGVCAGDGSTCRLVRGQS |
| | | | | KSHVSPEKREENVIAVPLGSRSVRITVKGPAHLFIESKTLQG |
| | | | | SKGEHSFNSPGVFLVENTTVEFQRGSERQTFKIPGPLMADFI |
| | | | | FKTRYTAAKDSVVQFFFYQPISHQWRQTDFFPCTVTCGGG |
| | | | | YQLNSAECVDIRLKRVVPDHYCHYYPENVKPKPKLKECS |
| | | | | MDPCPSSDGFKEIMPYDHFQPLPRWEHNPWTACSVSCGGG |
| | | | | IQRRSFVCVEESMHGEILQVEEWKCMYAPKPKVMQTCNL |
| | | | | FDCPKWIAMEWSQCTVTCGRGLRYRVVLCINHRGEHVGG |
| | | | | CNPQLKLHIKEECVIPIPCYKPKEKSPVEAKLPWLKQAQEL |
| | | | | EETRIATEEPTFIPEPWSACSTTCGPGVQVREVKCRVLLTFT |
| | | | | QTETELPEEECEGPKLPTERPCLLEACDESPASRELDIPLPE |
| | | | | DSETTYDWEYAGFTPCTATCVGGHQEAIAVCLHIQTQQTV |
| | | | | NDSLCDMVHRPPAMSQACNTEPCPPRWHVGSWGPCSATC |
| | | | | GVGIQTRDVYCLHPGETPAPPEECRDEKPHALQACNQFDC |
| | | | | PPGWHIEEWQQCSRTCGGGTQNRRVTCRQLLTDGSFLNLS |
| | | | | QRRKQVCQRLAAKGRRIPLSEMMCRDLPGLPLVRSCQMP |
| | | | | ECSKIKSEMKTKLGEQGPQILSVQRVYIQTREEKRINLTIGS |
| | | | | DELCQGPKASSHKSCARTDCPPHLAVGDWSKCSVSCGVGI |
| | | | | RAYLLPNTSVIIKCPVRRFQKSLIQWEKDGRCLQNSKRLGI |
| | | | | TKSGSLKIHGLAAPDIGVYRCIAGSAQETVVLKLIGTDNRLI |
| | | | | ARPALREPMREYPGMDHSEANSLGVTWHKMRQMWNNK |
| | | | | NDLYLDDDHISNQPFLRALLGHCSNSAGSTNSWELKNKQF |
| | | | | EAAVKQGAYSMDTAQFDELIRNMSQLMETGEVSDDLASQ |
| | | | | LIYQLVAELAKAQPTHMQWRGIQEETPPAAQLRGETGSVS |
| | | | | QSSHAKNSGKLTFKPKGPVLMRQSQPPSISFNKTINSRIGNT |
| | | | | VYITKRTEVINILCDLITPSEATYTWTKDGTLLQPSVKIILDG |
| | | | | TGKIQIQNPTRKEQGIYECSVANHLGSDVESSSVLYAEAPVI |
| | | | | LSVERNITKPEHNHLSVVVGGIVEAALGANVTIRCPVKGVP |
| | | | | QPNITWLKRGGSLSGNVSLLFNGSLLLQNVSLENEGTYVCI |
| | | | | ATNALGKAVATSVLHLLERRWPESRIVFLQGHKKYILQAT |
| | | | | NTRTNSNDPTGEPPPQEPFWEPGNWSHCSATCGHLGARIQ |
| | | | | RPQCVMANGQEVSEALCDHLQKPLAGFEPCNIRDCPARW |
| | | | | FTSVWSQCSVSCGEGYHSRQVTCKRTKANGTVQVVSPRA |
| | | | | CAPKDRPLGRKPCFGHPCVQWEPGNRCPGRCMGRAVRM |
| | | | | QQRHTACQHNSSDSNCDDRKRPTLRRNCTSGACDVCWHT |
| | | | | GPWKPCTAACGRGFQSRKVDCIHTRSCKPVAKRHCVQKK |
| | | | | KPISWRHCLGPSCDRDCTDTTHYCMFVKHLNLCSLDRYK |
| | | | | QRCCQSCQEG |

Table 5 identifies and defines the glycan structures included in Table 1. Table 5 identifies a graphical representation of the structure and a coded representation of the composition for each glycan structure included in Table 1. As used herein, the 4-digit GL NO. is a designation that represents the number of hexoses, the number of HexNAcs, the number of Fucoses, and the number of Neuraminic Acids.

TABLE 5
Glycan Structure GL NOS: Structure and Composition

| Glycan Structure | | |
|---|---|---|
| GL NO. | Glycan Symbol Structure | Glycan Composition |
| 1101 | 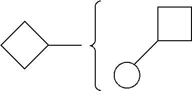 | Hex(1)HexNAc(1)Fuc(0)NeuAc(1) |
| 3400 | 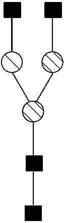 | Hex(3)HexNAc(4)Fuc(0)NeuAc(0) |
| 5401 | 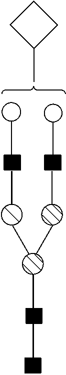 | Hex(5)HexNAc(4)Fuc(0)NeuAc(1) |
| 5402 | 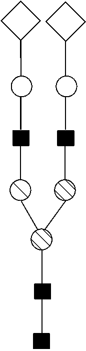 | Hex(5)HexNAc(4)Fuc(0)NeuAc(2) |
| 5411 | 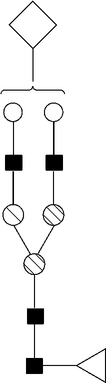 | Hex(5)HexNAc(4)Fuc(1)NeuAc(1) |
| 5412 | 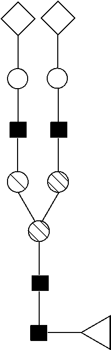 | Hex(5)HexNAc(4)Fuc(1)NeuAc(2) |
| 5510 | 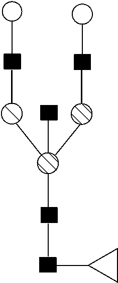 | Hex(5)HexNAc(5)Fuc(1)NeuAc(0) |
| 6301 | 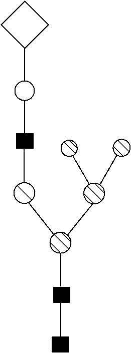 | Hex(6)HexNAc(3)Fuc(0)NeuAc(1) |
| 6503 | 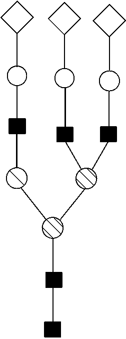 | Hex(6)HexNAc(5)Fuc(0)NeuAc(3) |
| 7602 | 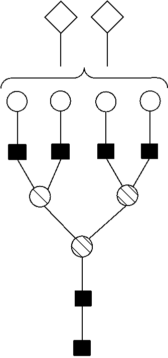 | Hex(7)HexNAc(6)Fuc(0)NeuAc(2) |
| 7603 | 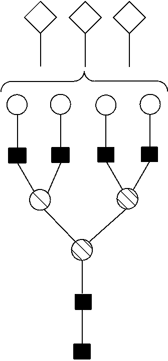 | Hex(7)HexNAc(6)Fuc(0)NeuAc(3) |
| 7614 | 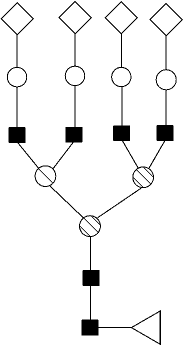 | Hex(7)HexNAc(6)Fuc(1)NeuAc(4) |

Legend for Table 5

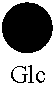

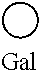

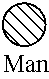

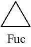

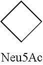

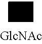

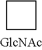

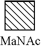

Table 5 illustrates the symbol structure and composition of detected glycan moieties that correspond to glycopeptides of Table 1 based on the Glycan GL NO. The term Symbol Structure illustrates a geometric linking structure of the carbohydrates where the bottommost carbohydrate such as N-acetylglucosamine is bound to the designated amino acid for an N-linked glycan and the rightmost carbohydrate such as N-acetylgalactosamine is bound to the designated amino acid for an O-linked glycan. For reference, N-linked glycans have a glycan attached to the amino acid asparagine and O-linked glycans have a glycan attached to either a serine or a threonine. It should be noted that glycan GL NO 1101 in Table 5 represents an O-linked glycan. All other glycans in 5 represent N-linked glycans.

The term Composition refers to the number of various classes of carbohydrates that make up the glycan. The quantity for each class of carbohydrate is depicted as a number in parenthesis to the right of an abbreviation that corresponds to the class of the carbohydrate. The abbreviations for these classes are Hex, HexNAc, Fuc, and NeuAc that respectively correspond to hexose, N-acetylhexosamine, fucose, and N-acetylneuraminic acid. It should be noted that hexose sugars include glucose, galactose, and mannose; and N-acetylhexosamine sugars includes N-acetylglucosamine, N-acetylgalactosamine, and N-acetylmannosamine. In various embodiments, the terms Neu5Ac, NeuAc, and N-acetylneuraminic acid may be referred to as sialic acid.

In some instances, a bracket symbol is used as part of the Symbol Structure (e.g., 7603) to indicate that the precise bonding linkage is not exactly known, but that the linking line segment is attached to one of the plurality of adjacent carbohydrates immediately adjacent to the bracket.

The identity of the various monosaccharides is illustrated by the Legend section located at the end of Table 5. The abbreviations of the Legend are Glc that represents glucose and is indicated by a dark circle, Gal that represents galactose and is indicated by an open circle, Man that represents mannose and is indicated by a circle with intermediate grey shading, Fuc that represents fucose and is indicated by a dark triangle, Neu5Ac that represents N-acetylneuraminic acid and is indicated by a dark diamond, GlcNAc that represents N-acetylglucosamine and is indicated by a dark square, GalNAc that represents N-acetylgalactosamine and is indicated by an open square, and ManNAc that represents N-acetylmannosamine and is indicated by a square with intermediate grey shading.

Aspects of the disclosure include kits comprising one or more compositions, each comprising one or more peptide structures of the disclosure that can be used as assay standards, and instructions for use. Kits in accordance with one or more embodiments described herein may include a label indicating the intended use of the contents of the kit. The term “label” as used herein with respect to a kit includes any writing, or recorded material supplied on or with a kit, or that otherwise accompanies a kit.

The peptide structures and the transitions produced therefrom, as described herein, may be useful for diagnosing and treating an sarcoma disease state. A transition includes a precursor ion and at least one product ion grouping. As reviewed herein, the peptide structures in Table 1, as well as their corresponding precursor ion and product ion groupings (these ions having defined m/z ratios or m/z ratios that fall within the m/z ranges identified herein), can be used in mass spectrometry-based analyses to diagnose and facilitate treatment of diseases, such as, for example, sarcoma.

Aspects of the disclosure include methods for analyzing one or more peptide structures, as described herein. In some embodiments, the methods involve processing a sample from a patient to generate a prepared sample that can be inputted into a mass spectrometry system (e.g., a reaction monitoring mass spectrometry system). In certain embodiments, processing the sample can comprise performing one or more of: a denaturation procedure, a reduction procedure, an alkylation procedure, and a digestion procedure. The denaturation and reduction procedures may be implemented in a manner similar to, for example, denaturation and reduction 202 in FIG. 2. The alkylation procedure may be implemented in a manner similar to, for example, alkylation procedure 204 in FIG. 2. The digestion procedure may be implemented in a manner similar to, for example, digestion procedure 206 in FIG. 2.

In some embodiments, the methods for analyzing one or more peptide structures involve detecting a set of product ions generated by a reaction monitoring mass spectrometry system in which one or more product ions may correspond to each of the one or more peptide structures that have been inputted into the mass spectrometry system. As described herein, each peptide structure can be converted into a set of product ions having a defined m/z ratio, as provided in Table 2 or an m/z ratio within an identified m/z ratio as provided in Table 2. In some embodiments, the methods involve generating quantification (e.g., abundance) data for the one or more product ions detected using the reaction monitoring mass spectrometry system.

In some embodiments, the methods further comprise generating a diagnosis output using the quantification data and a model that has been trained using supervised or unsupervised machine learning. In certain embodiments, the reaction monitoring mass spectrometry system may include multiple/selected reaction monitoring mass spectrometry (MRM/SRM-MS) to detect the one or more product ions and generate the quantification data.

### VII. Exemplary Embodiments for Managing Sarcoma Treatment

The present disclosure concerns embodiments for systems, methods, and compositions related to managing treatment for sarcoma. The embodiments concern classifying biological samples, measuring for one or more certain markers from a biological sample, assaying for one or more certain markers from a biological sample, determining the presence of one or more certain markers from a biological sample, and so forth. The embodiments of the disclosure utilize models that accurately identify that an individual will or will not respond to sarcoma therapy based on the presence of one or more markers in sample(s) from the individual. In some embodiments, the markers are accurate regardless of the status of one or more characteristics of the individual: biological sex, sample source, sample collection, smoker status, or age.

In various embodiments of the disclosure, an individual is in need of identifying whether or not they will respond to a sarcoma therapy of any kind, including immunotherapies, such as antibodies. In some cases, the analysis of the sample of the individual is the sole test utilized for identifying treatment outcome, whereas in other cases a medical provider may utilize one or more other tests.

Any individual may be subject to methods of the disclosure, including any person of any biological sex, any gender, any smoker status, any ethnicity, and so forth. In various embodiments, an individual is subject to any method encompassed herein as a part of routine preventative or health check medical practices or because sarcoma is present or suspected of being present (such as having a family history).

In particular embodiments, the sample for analysis for managing a treatment outcome is serum from the individual. The present disclosure provides for measuring for one or more circulating glycoproteins, glycopeptides, or non-glycosylated peptides in serum to determine the likelihood that an individual will respond to treatment. In various embodiments, the sample is measured for 1, 2, 3, 4, 5, 6, 7, 8, 9, 10, 11, 12, 13, 14, 15, 16, 17, 18, 19, 20, 21, or all 22 of the peptides of Table 1.

In particular embodiments, the disclosure encompasses methods of measuring levels of one or more of PS-1, PS-2, PS-3, PS-4, PS-5, PS-6, PS-7, PS-8, PS-9, PS-10, PS-11, PS-12, PS-13, PS-14, PS-15, PS-16, PS-17, PS-18, PS-19, PS-20, PS-21, and PS-22 of Table 1 from a sample from an individual in need of sarcoma therapy such that upon determination of the level(s) from the sample, the individual is administered an effective amount of a particular sarcoma therapy, including one or more antibodies. In particular embodiments, the disclosure encompasses methods of measuring levels of one or more of PS-1, PS-2, PS-3, PS-4, PS-5, PS-6, PS-7, PS-8, PS-9, PS-10, PS-11, PS-12, PS-13, PS-14, PS-15, PS-16, PS-17, PS-18, PS-19, PS-20, PS-21, and PS-22 of Table 1 from a sample from an individual in need of sarcoma therapy such that upon determination of the level(s) from the sample, the individual is not administered the particular sarcoma therapy. In specific cases, there are measurements over a selected threshold from a sample from an individual of one or more of PS-1, PS-2, PS-3, PS-4, PS-5, PS-6, PS-7, PS-8, PS-9, PS-10, PS-11, PS-12, PS-13, PS-14, PS-15, PS-16, PS-17, PS-18, PS-19, PS-20, PS-21, and PS-22 of Table 1 and the individual is administered a certain sarcoma therapy. In specific cases, there are measurements over a selected threshold from a sample from an individual of one or more of PS-1, PS-2, PS-3, PS-4, PS-5, PS-6, PS-7, PS-8, PS-9, PS-10, PS-11, PS-12, PS-13, PS-14, PS-15, PS-16, PS-17, PS-18, PS-19, PS-20, PS-21, and PS-22 of Table 1 and the individual is not administered a certain sarcoma therapy.

Embodiments of the disclosure include methods of classifying samples, including serum samples, from an individual suspected of being a non-responder to one or more sarcoma treatments, or at risk for being a non-responder to one or more sarcoma treatments, by measuring from the sample for one or more glycopeptides and/or non-glycosylated peptides encompassed herein. The methods encompass determination of whether or not a sarcoma therapy will be therapeutic in the individual. In some cases, the measuring identifies or predicts that the individual will not have a response to a future therapy or the measuring identifies or predicts that the individual will have a response to a future therapy. In various embodiments, in cases wherein the individual has one or more glycopeptides and/or non-glycosylated peptides of Table 1, or certain levels thereof compared to control or healthy individuals, the individual may be determined to be a responder to the therapy. In various embodiments, in cases wherein the individual lacks one or more glycopeptides and/or non-glycosylated peptides of Table 1, or has certain levels thereof compared to control or healthy individuals, the individual may be determined not to be a responder to the therapy.

In embodiments wherein the measuring identifies the individual as being a responder to the therapy, the individual may be recommended to take an effective amount of the therapy, such as the therapy being an immunotherapy (including one or more antibodies), radiation, chemotherapy, etc. In embodiments wherein the measuring identifies the individual as being a responder to the therapy, the individual may be recommended to take an effective amount of a different therapy.

### VIII. Representative Experimental Results

### VIII.A. Sample Population-Cohort

Survival information and biological samples were collected for total of 43 patients (sample subjects) diagnosed with sarcoma (bone and soft tissue sarcoma) of various morphologies and topologies were obtained from the Garvan Institute. These samples were collected at a baseline point in time just prior to treatment. The treatment comprised a combination of durvalumab and tremelimumab. The samples were collected from various sources, stored at −80° C. (Celsius), processed and analyzed through a workflow such as workflow 100 described with respect to FIGS. 1, 2A, and 2B. The samples were processed via MRM analysis to generate quantification data for a panel of 589 peptide structures (519 glycopeptide structures and 80 aglycosylated peptide structures).

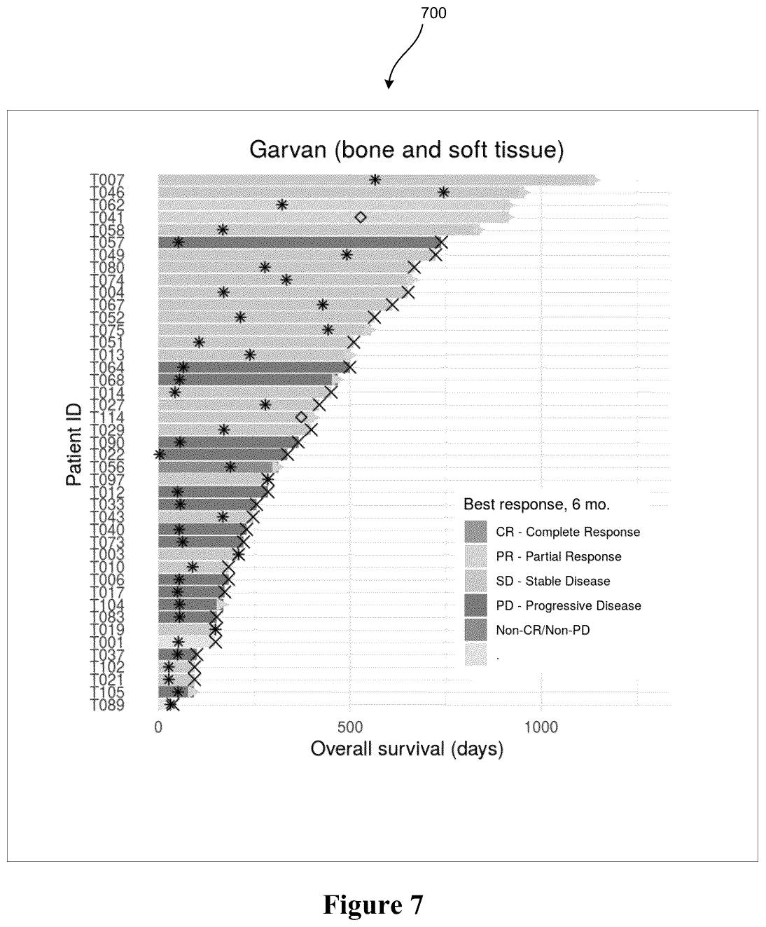

FIG. 7 is a graph describing the survival information for the 43-patient cohort in accordance with one or more embodiments. Graph 700 includes various markers that indicate an overall survival (OS) censor, death, progression-free survival (PFS) time, and PFS censor. The OS censor marker indicates that death was not observed for the subject and indicates the time of last contact or last clinical assessment relative to the baseline point in time. The death marker indicates the time of death relative to the baseline point in time. The PFS time marker indicates the time at which a non-death disruption event occurred relative to the baseline point in time. For example, the non-death disruption event may be any event evidencing advancement or progression of the sarcoma disease state. The PFS censor marker may indicate, for example, the time of last clinical assessment at which the patient could be characterized as being progression-free relative to the baseline point in time.

### VIII.B. Identification of Peptide Structures for Final Model

As described above, a panel consisting of 589 peptide structures was assessed for the sample population. Age and sex adjusted Cox regression analysis (specifically, Cox PH regression analysis) was performed on a peptide structure by peptide structure basis with overall survival (time to death relative to baseline point in time (e.g., baseline blood draw, immunotherapy start time, etc.)) serving as the primary independent variable. Exponentiated coefficients (hazard ratios, or HRs) were generated and interpreted as the multiplicative change in the risk of death when peptide structure expression increases by 1 unit, adjusted for sex and age. Additionally, the analysis corrected for making a comparison for each of the 589 markers simultaneously. P-values and false discovery rates (FDRs) were also computed.

Table 6 below lists the HRs, P-values, and FDRs for a selected number of the most significant peptide structures based on p-values. These peptide structures are also identified in Table 1 above. As shown in Table 6, for the purposes of this analysis, any quantification data for PS-2 and PS-3 were treated as being for the same marker. Any quantification data for PS-15 and PS-16 were treated as being for the same marker. Any quantification data for PS-19 and PS-20 were treated as being for the same marker.

TABLE 6
Peptide Structures and COX Values

| PS-ID NO. | COX PH HR | COX PH P-value | COX PH FDR |
|---|---|---|---|
| PS-1 | 1.829 | 0.0031 | 0.465 |
| PS-2 and PS-3 | 2.308 | 0.0040 | 0.465 |
| PS-4 | 2.067 | 0.0047 | 0.465 |
| PS-5 | 1.988 | 0.0062 | 0.465 |
| PS-6 | 1.937 | 0.0063 | 0.465 |
| PS-7 | 0.569 | 0.0074 | 0.465 |
| PS-8 | 1.745 or 1.673 | 0.00987 or 0.0127 | 0.465 |
| PS-9 | 1.81 | 0.0106 | 0.465 |
| PS-10 | 1.699 | 0.0114 | 0.465 |
| PS-11 | 1.593 | 0.0120 | 0.465 |
| PS-12 | 1.81 | 0.0122 | 0.465 |
| PS-13 | 0.732 | 0.0124 | 0.465 |
| PS-14 | 1.63 | 0.0129 | 0.465 |
| PS-15 and PS-16 | 1.909 | 0.0134 | 0.465 |
| PS-17 | 1.67 | 0.0147 | 0.465 |
| PS-18 | 0.585 | 0.0151 | 0.465 |
| PS-19 and PS-20 | 1.754 | 0.0157 | 0.465 |
| PS-21 | 0.607 | 0.0165 | 0.465 |
| PS-22 | 1.666 | 0.0167 | 0.465 |

A LOOCV Cox regression algorithm with ridge penalty was used to build the final model. The LOOCV Cox regression algorithm included building a model for each patient by fitting the Cox regression model to the data of the other 42 patients. The model built for a given patient was then used to predict a response score for each patient to generate a distribution of response scores. This distribution was then processed to identify the cutoff response score that maximized Harrell's C-index to dichotomize the likely responders and likely non-responders. A HR and p-value were computed for the model. This process was iterated to generate multiple models up to a ceiling (sample size/5 or sample size/4) to prevent overfitting of models. Each iteration i used the i^{th} most significant peptide structures with respect to the p-values identified above in Table 6 to build the model for that iteration. Ten models were generated for a ceiling of 10 (e.g., 43 divided by 4).

Table 7 below provides the HRs and p-values for the eight models generated. The peptide structure that a cumulative HR and P-value is associated with is the peptide structure that was added to the set for that particular iteration. For example, in Table 7, the cumulative HR and cumulative p-value for PS-3 are for the model built using PS1 through PS-3. As another example, the cumulative HR and cumulative p-value for PS-5 are for the model built using PS1 through PS-5. As shown in Table 7, for the purposes of this analysis, any quantification data for PS-2 and PS-3 were treated as being for the same marker and thus these two peptide structures were considered as a single marker.

TABLE 7
LOOCV - Cumulative HRs and P-values

| PS-ID NO. | LOOCV Cumulative HR | LOOCV Cumulative P-value |
|---|---|---|
| PS-1 | 4.12 | 0.0006000 |
| PS-2 and PS-3 | 8.216 | 0.0000210 |
| PS-4 | 2.992 | 0.0097400 |
| PS-5 | 6.461 | 0.0001220 |
| PS-6 | 3.015 | 0.0050500 |
| PS-7 | 4.036 | 0.0020100 |
| PS-8 | 2.563 | 0.0125000 |
| PS-9 | 3.418 | 0.0017900 |
| PS-10 | 3.092 | 0.0046700 |
| PS-11 | 2.748 | 0.0167000 |

Based on the cumulative HRs and p-values, a final model was selected. The final model used PS-1 through PS-3, where PS-2 and PS-3 were considered as a single marker.

### VIII.C. Validation and Results

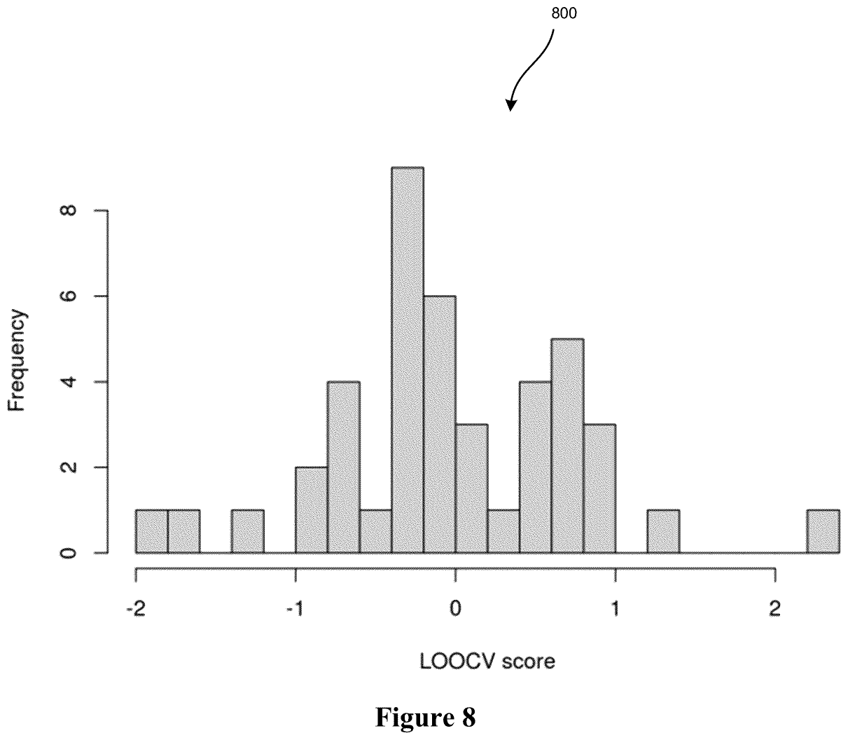

FIG. 8 is a plot 800 of the distribution of the response scores generated for the final model described above in accordance with one or more embodiments.

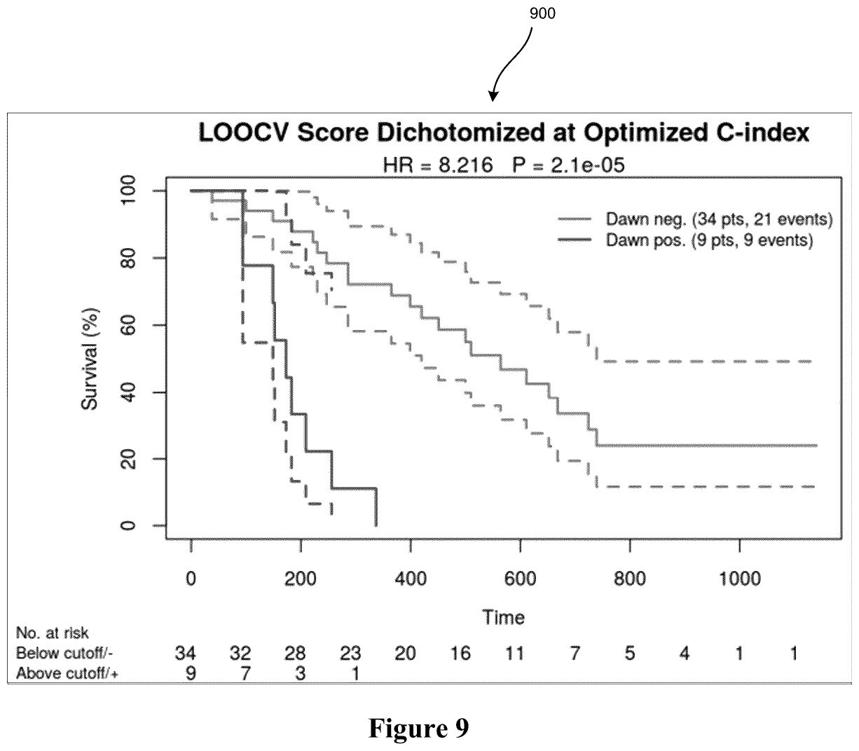

FIG. 9 is a plot 900 of time versus percentage of survival in accordance with one or more embodiments. Plot 900 indicates the number of patients predicted to be likely non-responders, those at risk of death, as those number of patients having a response score above the selected threshold with respect to a given point in time. Plot 900 also indicates the number of patients predicted to be likely responders as those number of patients having a response score below the selected threshold with respect to a given point in time and thus predicted to survive beyond the given point in time.

For example, at 0 days, 9 patients were predicted to be likely non-responders and are thus at risk of death, and 34 patients were predicted to be likely responders. At 200 days, 3 patients of 9 who were predicted to be likely non-responders are still alive and thus at risk of death, while 28 patients of 34 who were predicted to be likely responders had survived to 200 days and remain at risk of death.

Further, plot 900 includes two Kaplan-Meier (KM) curves 902 and 904 for the dichotomized predicted response score. Curve 902 represents patients predicted to be likely responders. Curve 904 represents patients predicted to be likely non-responders. Every step-down (or drop) in curve 902 and curve 904 represents a patient's death, where the size of the step-down is inversely proportional to how many patients are still at risk of death in that particular group.

### IX. Recitation of Embodiments

Embodiment 1: A method for managing a treatment of a subject diagnosed with a sarcoma disease state, the method comprising: receiving peptide structure data corresponding to a biological sample obtained from the subject; computing a response score that predicts a likelihood of responsiveness to the treatment using quantification data identified from the peptide structure data for a set of peptide structures, wherein the set of peptide structures includes at least one peptide structure identified from a plurality of peptide structures listed in Table 1; and wherein the plurality of peptide structures is listed in Table 1 with respect to relative significance to a survival for the sarcoma disease state; and generating a treatment response output based on the response score.

Embodiment 2: The method of Embodiment 1, wherein generating the treatment response output comprises: determining whether the response score is above a selected threshold; identifying the subject as a likely responder to the treatment when the response score is above the selected threshold; and identifying the subject as a likely non-responder to the treatment when the response score is not above the selected threshold

Embodiment 3: The method of Embodiment 1, wherein generating the treatment response output comprises: determining whether the response score is above a selected threshold; identifying the subject as a likely responder to the treatment when the response score is either at or above the selected threshold; and identifying the subject as a likely non-responder to the treatment when the response score is below the selected threshold.

Embodiment 4: The method of Embodiment 2 or Embodiment 3, wherein the selected threshold is a cutoff response score that maximizes a concordance index.

Embodiment 5: The method of Embodiment 4, wherein the cutoff response score and the concordance index are computed using a distribution of sample response scores generated for a plurality of sample subjects.

Embodiment 6: The method of Embodiment 1, wherein the response score is a probability that the subject will respond to the treatment based on a set of survival criteria.

Embodiment 7: The method of Embodiment 6 wherein the set of survival criteria includes an overall survival greater than a selected period of time.

Embodiment 8: The method of Embodiment 6, wherein the set of survival criteria includes a progression free survival greater than a selected period of time.

Embodiment 9: The method of any one of Embodiments 1-8, wherein computing the response score comprises: computing the response score using a Cox regression model and the quantification data.

Embodiment 10: The method of any one of Embodiments 1-9, wherein the at least one peptide structure comprises a glycopeptide structure defined by a peptide sequence and a glycan structure linked to the peptide sequence at a linking site of the peptide sequence, as identified in Table 1, with the peptide sequence being one of SEQ ID NOS: 12-26 as defined in Table 1.

Embodiment 11: The method of any one of Embodiments 1-10, further comprising: determining a selected threshold for the response score to use in generating the treatment response output.

Embodiment 12: The method of Embodiment 11, wherein determining the selected threshold comprises: generating a distribution of sample response scores for a plurality of sample subjects diagnosed with the sarcoma disease state using a selected portion of sample data corresponding to the set of peptide structures for a plurality of sample subjects and survival information for the plurality of sample subjects; and identifying a cutoff response score that maximizes a concordance index for the distribution of samples response scores as the selected threshold.

Embodiment 13: The method of Embodiment 12, further comprising: performing a Cox regression analysis for each peptide structure in the plurality of peptide structure profiles for the plurality of sample subjects; identifying, based on the Cox regression analysis, an initial group of peptide structures that is associated with survivability for the sarcoma disease state; and forming the selected portion of the sample data based on the initial group of peptide structures identified.

Embodiment 14: The method of Embodiment 13, wherein identifying, based on the Cox regression analysis, the initial group of peptide structures comprises: identifying a selected number of most significant peptide structures with respect to p-values as the initial group of peptide structures.

Embodiment 15: The method of Embodiment 13 or Embodiment 14, wherein computing the response score comprises: computing the response score using a model built for a subset of peptide structures selected from the initial group of peptide structures.

Embodiment 16: The method of Embodiment 15, wherein the subset of peptide structures includes at least one of PS-1 through PS-5 in Table 1.

Embodiment 17: The method of any one of Embodiments 1-16, wherein the quantification data for a peptide structure of the set of peptide structures comprises at least one of an abundance, a relative abundance, a normalized abundance, a relative quantity, an adjusted quantity, a normalized quantity, a relative concentration, an adjusted concentration, or a normalized concentration.

Embodiment 18: The method of any one of Embodiments 1-17, wherein the peptide structure data is generated using multiple reaction monitoring mass spectrometry (MRM-MS).

Embodiment 19: The method of any one of Embodiments 1-18, further comprising:
creating a sample from the biological sample; and preparing the sample using reduction, alkylation, and enzymatic digestion to form a prepared sample that includes a set of peptide structures.

Embodiment 20: The method of Embodiment 19, further comprising: generating the peptide structure data from the prepared sample using multiple reaction monitoring mass spectrometry (MRM-MS).

Embodiment 21: The method of any one of Embodiments 1-20 wherein generating the treatment response output comprises: generating a report that indicates that the subject will be a likely responder to the treatment for the sarcoma disease state.

Embodiment 22: The method of Embodiment 21, wherein the report comprises an indication to proceed with administration of the treatment to the subject.

Embodiment 23: The method of any one of Embodiments 1-22, further comprising: administering a therapeutic dosage of the treatment based on the treatment response output indicating that the subject will be a likely responder to the treatment.

Embodiment 24: The method of Embodiment 23, wherein the therapeutic dosage comprises: 1500 mg durvalumab via intravenous (IV) infusion every 4 weeks for up to 4 doses; 75 mg tremelimumab via IV infusion every 4 weeks for up to 4 doses; and 1500 mg durvalumab every 4 weeks starting on Week 16 for up to 9 doses.

Embodiment 25: The method of any one of Embodiments 1-20, further comprising: generating a report that indicates that the subject will be a likely non-responder to the treatment for the sarcoma disease state.

Embodiment 26: The method of Embodiment 25, wherein the report comprises an indication to either modify the treatment or select a new treatment.

Embodiment 27: The method of any one of Embodiments 1-25, wherein the treatment comprises an immunotherapy.

Embodiment 28: The method of any one of Embodiments 1-26, wherein the treatment comprises durvalumab and tremelimumab.

Embodiment 29: The method of any one of Embodiments 1-26, further comprising:

sending the treatment output to a remote system.

Embodiment 30: A method of building a final model to predict treatment responsiveness for a subject diagnosed with a sarcoma disease state, the method comprising: receiving sample data for a panel of peptide structures for a plurality of sample subjects diagnosed with the sarcoma disease state, the sample data comprising quantification data for the panel of peptide structures; receiving survival information for the plurality of sample subjects; identifying, based on the sample data and the survival information, an initial group of peptide structures that are associated with survival of the sarcoma disease state, wherein the initial group of peptide structures includes at least 3 peptide structures of a plurality of peptide structures identified in Table 1; building a plurality of models using different subsets of the initial group of peptide structures; and selecting the final model from the plurality of models for use in predicting the treatment responsiveness for the treatment to sarcoma.

Embodiment 31: The method of Embodiment 30, wherein the final model is a Cox regression model.

Embodiment 32: The method of Embodiment 30 or Embodiment 31, wherein identifying, based on the sample data and the survival information, the initial group of peptide structures comprises: performing a Cox regression analysis for each peptide structure in the plurality of peptide structure profiles for the plurality of sample subjects; computing p-values for each peptide structure; and identifying, based on the Cox regression analysis, the initial group of peptide structures that are associated with survival with respect to the sarcoma disease state based on the p-values.

Embodiment 33: The method of any one of Embodiments 30-32, wherein building the plurality of models comprises: selecting a test subset of peptide structures from the initial group of peptide structures to build a model; and generating a distribution of sample response scores for the plurality of sample subjects using the model, the survival information, and a portion of the sample data for the plurality of sample subjects corresponding to the test subset of peptide structures for the plurality of sample subjects.

Embodiment 34: The method of Embodiment 33, wherein building the plurality of models further comprises: determining a selected threshold for the model based on the distribution; and computing a hazard ratio and a p-value for the model.

Embodiment 35: The method of any one of Embodiments 30-34, wherein selecting the final model comprises: selecting the final model from the plurality of models generated based on a plurality of p-values computed for the plurality of models.

Embodiment 36: The method of any one of Embodiments 30-35, wherein the final model uses a set of peptide structures that includes least one of PS-1 through PS-5 in Table 1.

Embodiment 37: The method of any one of Embodiments 30-36, wherein the quantification data for the panel of peptide structures for the plurality of subjects diagnosed with the plurality of sarcoma disease states comprises at least one of an abundance, a relative abundance, a normalized abundance, a relative quantity, an adjusted quantity, a normalized quantity, a relative concentration, an adjusted concentration, or a normalized concentration.

Embodiment 38: A method of treating sarcoma in a patient, comprising: receiving peptide structure data corresponding to a biological sample obtained from the patient; computing a response score that predicts a likelihood of responsiveness to a treatment using quantification data identified from the peptide structure data for a set of peptide structures, wherein the set of peptide structures includes at least one peptide structure identified from a plurality of peptide structures listed in Table 1; wherein the plurality of peptide structures is listed in Table 1 with respect to relative significance to a survival for the sarcoma disease state; determining whether the subject is a likely responder or a likely non-responder for the treatment based on the response score; and administering a therapeutic dosage of the treatment to the patient if the subject is determined to be the likely responder.

Embodiment 39: The method of Embodiment 38, wherein administering the treatment comprises: administering an immunotherapy to the patient.

Embodiment 40: The method of Embodiment 38, wherein administering the treatment comprises: administering a combination of durvalumab and tremelimumab to the patient.

Embodiment 41: The method of Embodiment 38, wherein administering the treatment comprises: administering the durvalumab at a dosage of 1500 mg via intravenous (IV) route of administration every 4 weeks for up to 4 doses.

Embodiment 42: The method of Embodiment 38, wherein administering the treatment comprises: administering the durvalumab at a dosage of 1500 mg via intravenous (IV) route of administration every 4 weeks for up to 13 doses.

Embodiment 43: The method of Embodiment 38, wherein administering the treatment comprises: administering the durvalumab at a dosage of 1500 mg via intravenous (IV) route of administration every 4 weeks starting at week 16 for up to 9 doses.

Embodiment 44: The method of Embodiment 38, wherein administering the treatment comprises: administering the tremelimumab at a dosage of 75 mg via intravenous (IV) route of administration every 4 weeks starting for up to 4 doses.

20) Embodiment 45: The method of Embodiment 38, wherein administering the treatment comprises: administering the treatment via intravenous (IV) route of administration.

Embodiment 46: The method of Embodiment 38, wherein administering the treatment comprises: administering the therapeutic dosage of the treatment every 4 weeks.

Embodiment 47: A composition comprising at least one of peptide structures PS-1 to PS-22 identified in Table 1.

Embodiment 48: A composition comprising a peptide structure or a product ion, wherein: the peptide structure or product ion comprises an amino acid sequence having at least 90% sequence identity to any one of SEQ ID NOS: 12-26, corresponding to peptide structures PS-1 to PS-22 in Table 1; and the product ion is selected as one from a group consisting of product ions identified in Table 2 including product ions falling within an identified m/z range.

Embodiment 49: A composition comprising a glycopeptide structure selected as one from a group consisting of peptide structures PS-1 to PS-22 identified in Table 1, wherein:
the glycopeptide structure comprises: an amino acid peptide sequence identified in Table 3 as corresponding to the glycopeptide structure; and a glycan structure identified in

Table 5 as corresponding to the glycopeptide structure in which the glycan structure is linked to a residue of the amino acid peptide sequence at a corresponding position identified in Table 1; and wherein the glycan structure has a glycan composition.

Embodiment 50: The composition of Embodiment 48, wherein the glycan composition is identified in Table 5.

Embodiment 51: The composition of Embodiment 48 or Embodiment 50, wherein: the glycopeptide structure has a precursor ion having a charge identified in Table 2 as corresponding to the glycopeptide structure.

Embodiment 52: The composition of any one of Embodiments 48-51, wherein: the glycopeptide structure has a precursor ion with an m/z ratio within +1.5 of the m/z ratio listed for the precursor ion in Table 2 as corresponding to the glycopeptide structure.

Embodiment 53: The composition of any one of Embodiments 48-51, wherein: the glycopeptide structure has a precursor ion with an m/z ratio within +1.0 of the m/z ratio listed for the precursor ion in Table 2 as corresponding to the glycopeptide structure.

Embodiment 54: The composition of any one of Embodiments 44-51, wherein: the glycopeptide structure has a precursor ion with an m/z ratio within +0.5 of the m/z ratio listed for the precursor ion in Table 2 as corresponding to the glycopeptide structure.

Embodiment 55: The composition of any one of Embodiments 48-54, wherein: the glycopeptide structure has a product ion with an m/z ratio within +1.0 of the m/z ratio listed for the first product ion in Table 2 as corresponding to the glycopeptide structure.

Embodiment 56: The composition of any one of Embodiments 48-54, wherein: the glycopeptide structure has a product ion with an m/z ratio within +0.8 of the m/z ratio listed for the first product ion in Table 2 as corresponding to the glycopeptide structure.

Embodiment 57: The composition of any one of Embodiments 48-54, wherein: the glycopeptide structure has a product ion with an m/z ratio within +0.5 of the m/z ratio listed 25 for the first product ion in Table 2 as corresponding to the glycopeptide structure.

Embodiment 58: The composition of any one of Embodiments 48-54, wherein the glycopeptide structure has a monoisotopic mass identified in Table 1 as corresponding to the glycopeptide structure.

Embodiment 59: A composition comprising a peptide structure selected as one from a plurality of peptide structures identified in Table 1, wherein: the peptide structure has a monoisotopic mass identified as corresponding to the peptide structure in Table 1; and the peptide structure comprises the amino acid sequence of SEQ ID NOs: 12-26 identified in Table 1 as corresponding to the peptide structure.

Embodiment 60: The composition of Embodiment 59, wherein: the peptide structure has a precursor ion having a charge identified in Table 2 as corresponding to the peptide structure.

Embodiment 61: The composition of Embodiment 59 or Embodiment 60, wherein: the peptide structure has a precursor ion with an m/z ratio within +1.5 of the m/z ratio listed for the precursor ion in Table 2 as corresponding to the peptide structure.

Embodiment 62: The composition of Embodiment 59 or Embodiment 60, wherein: the peptide structure has a precursor ion with an m/z ratio within +1.0 of the m/z ratio listed for the precursor ion in Table 2 as corresponding to the peptide structure.

Embodiment 63: The composition of Embodiment 59 or cl Embodiment aim 60, wherein: the peptide structure has a precursor ion with an m/z ratio within +0.5 of the m/z ratio listed for the precursor ion in Table 2 as corresponding to the peptide structure.

Embodiment 64: The composition of any one of Embodiments 59-63, wherein: the peptide structure has a product ion with an m/z ratio within +1.0 of the m/z ratio listed for the first product ion in Table 2 as corresponding to the peptide structure.

Embodiment 65: The composition of any one of Embodiments 59-63, wherein: the peptide structure has a product ion with an m/z ratio within +0.8 of the m/z ratio listed for the first product ion in Table 2 as corresponding to the peptide structure.

Embodiment 66: The composition of any one of Embodiments 59-63, wherein: the peptide structure has a product ion with an m/z ratio within +0.5 of the m/z ratio listed for the first product ion in Table 2 as corresponding to the peptide structure.

Embodiment 67: A kit comprising at least one agent for quantifying at least one peptide structure identified in Table 1 to carry out the method of any one of Embodiments 1-46.

Embodiment 68: A kit comprising at least one of a glycopeptide standard, a buffer, or a set of peptide sequences to carry out the method of any one of Embodiments 1-37, a peptide sequence of the set of peptide sequences identified by a corresponding one of SEQ ID NOS: 12-26, defined in Table 1.

Embodiment 69: A system comprising: one or more data processors; and a non-transitory computer readable storage medium containing instructions which, when executed on the one or more data processors, cause the one or more data processors to perform part or all of any one of Embodiments 1-46.

Embodiment 70: A computer-program product tangibly embodied in a non-transitory machine-readable storage medium, including instructions configured to cause one or more data processors to perform part or all of any one of Embodiments 1-46.

Embodiment 71: The method of Embodiment 6, wherein the glycan structure corresponds to a glycan structure GL number in accordance with Table 1, wherein the glycan structure comprises a symbol structure in accordance with the glycan structure GL number of Table 1 and Table 5.

Embodiment 72: The method of Embodiment 6, wherein the glycan structure corresponds to a glycan structure GL number in accordance with Table 1, wherein the glycan structure comprises a glycan composition in accordance with the glycan structure GL number of Table 1 and Table 5.

### X. Additional Considerations

Any headers and/or sub-headers between sections and subsections of this document are included solely for the purpose of improving readability and do not imply that features cannot be combined across sections and subsection. Accordingly, sections and subsections do not describe separate embodiments.

While the present teachings are described in conjunction with various embodiments, it is not intended that the present teachings be limited to such embodiments. On the contrary, the present teachings encompass various alternatives, modifications, and equivalents, as will be appreciated by those of skill in the art. The present description provides preferred exemplary embodiments, and is not intended to limit the scope, applicability or configuration of the disclosure. Rather, the present description of the preferred exemplary embodiments will provide those skilled in the art with an enabling description for implementing various embodiments.

It is understood that various changes may be made in the function and arrangement of elements without departing from the spirit and scope as set forth in the appended claims. Thus, such modifications and variations are considered to be within the scope set forth in the appended claims. Further, the terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed.

In describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

Specific details are given in the present description to provide an understanding of the embodiments. However, it is understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

1. A method for managing a treatment of a subject diagnosed with a sarcoma disease state, the method comprising:
receiving peptide structure data corresponding to a biological sample obtained from the subject;
computing a response score that predicts a likelihood of responsiveness to the treatment using quantification data identified from the peptide structure data for a set of peptide structures,
wherein the set of peptide structures includes at least one peptide structure identified from a plurality of peptide structures listed in Table 1, and
wherein the plurality of peptide structures is listed in Table 1 with respect to relative significance to a survival for the sarcoma disease state; and
generating a treatment response output based on the response score.

2. (canceled)

3. The method of claim 1, wherein the generating of the treatment response output comprises:
determining whether the response score is above a selected threshold;
identifying the subject as a likely responder to the treatment when the response score is either at or above the selected threshold; and
identifying the subject as a likely non-responder to the treatment when the response score is below the selected threshold.

4. The method of claim 23, wherein the selected threshold is a cutoff response score that maximizes a concordance index.

5.-8. (canceled)

9. The method of claim 1, wherein the computing of the response score comprises:
computing the response score using a Cox regression model and the quantification data.

10. The method of claim 1, wherein the at least one peptide structure comprises a glycopeptide structure defined by a peptide sequence and a glycan structure linked to the peptide sequence at a linking site of the peptide sequence, as identified in Table 1, with the peptide sequence being one of SEQ ID NOS: 12-26 as defined in Table 1.

11. (canceled)

12. The method of claim 1, further comprising:
generating a distribution of sample response scores for a plurality of sample subjects diagnosed with the sarcoma disease state using a selected portion of sample data corresponding to the set of peptide structures for a plurality of sample subjects and survival information for the plurality of sample subjects; and
identifying a cutoff response score that maximizes a concordance index for the distribution of samples response scores as a selected threshold for the response score in generating the treatment response output.

13. The method of claim 12, further comprising:
performing a Cox regression analysis for each peptide structure in the plurality of peptide structures for the plurality of sample subjects;
identifying, based on the Cox regression analysis, an initial group of peptide structures that is associated with survivability for the sarcoma disease state; and
forming the selected portion of the sample data based on the initial group of peptide structures identified.

14. The method of claim 13, wherein the identifying, based on the Cox regression analysis, of the initial group of peptide structures comprises:
identifying a selected number of most significant peptide structures with respect to p-values as the initial group of peptide structures.

15. The method of claim 13, wherein the computing of the response score comprises:
computing the response score using a model built for a subset of peptide structures selected from the initial group of peptide structures,
wherein the subset of peptide structures includes at least one of PS-1 through PS-5 in Table 1.

16. (canceled)

17. The method of claim 1, wherein the quantification data for a peptide structure of the set of peptide structures comprises at least one of an abundance, a relative abundance, a normalized abundance, a relative quantity, an adjusted quantity, a normalized quantity, a relative concentration, an adjusted concentration, or a normalized concentration.

18. The method of claim 1, wherein the peptide structure data is generated using multiple reaction monitoring mass spectrometry (MRM-MS).

19.-22. (canceled)

23. The method of claim 1, further comprising:
administering a therapeutic dosage of the treatment based on the treatment response output indicating that the subject will be a likely responder to the treatment,
wherein the therapeutic dosage comprises
1500 mg durvalumab via intravenous (IV) infusion every 4 weeks for up to 4 doses;
75 mg tremelimumab via IV infusion every 4 weeks for up to 4 doses; and
1500 mg durvalumab every 4 weeks starting on Week 16 for up to 9 doses.

24.-29. (canceled)

30. A method of building an optimized model to predict treatment responsiveness for a subject diagnosed with a sarcoma disease state, the method comprising:
receiving sample data for a panel of peptide structures for a plurality of sample subjects diagnosed with the sarcoma disease state, the sample data comprising quantification data for the panel of peptide structures;
receiving survival information for the plurality of sample subjects;
identifying, based on the sample data and the survival information, an initial group of peptide structures that are associated with survival of the sarcoma disease state, wherein the initial group of peptide structures includes at least 3 peptide structures of a plurality of peptide structures identified in Table 1;
building a plurality of models using different subsets of the initial group of peptide structures; and
selecting the optimized model from the plurality of models for predicting the treatment responsiveness for the treatment to sarcoma.

31. (canceled)

32. The method of claim 30, wherein the identifying, based on the sample data and the survival information, of the initial group of peptide structures comprises:
performing a Cox regression analysis for each peptide structure in the plurality of peptide structures for the plurality of sample subjects;
computing p-values for each peptide structure; and
identifying, based on the Cox regression analysis, the initial group of peptide structures that are associated with survival with respect to the sarcoma disease state based on the p-values.

33. The method of claim 30, wherein the building of the plurality of models comprises:
selecting a test subset of peptide structures from the initial group of peptide structures to build a model of the plurality of models;
generating a distribution of sample response scores for the plurality of sample subjects using the model, the survival information, and a portion of the sample data for the plurality of sample subjects corresponding to the test subset of peptide structures for the plurality of sample subjects;
determining a selected threshold for the model based on the distribution; and
computing a hazard ratio and a p-value for the model.

34. (canceled)

35. The method of claim 30, wherein the selecting of the optimized model comprises:
selecting the optimized final-model from the plurality of models generated based on a plurality of p-values computed for the plurality of models.

36. The method of claim 30, wherein the optimized model uses a set of peptide structures that includes at least one of PS-1 through PS-5 in Table 1.

37. (canceled)

38. A method of treating sarcoma in a patient, comprising:
receiving peptide structure data corresponding to a biological sample obtained from the patient;
computing a response score that predicts a likelihood of responsiveness to a treatment using quantification data identified from the peptide structure data for a set of peptide structures, wherein the set of peptide structures includes at least one peptide structure identified from a plurality of peptide structures listed in Table 1;
wherein the plurality of peptide structures is listed in Table 1 with respect to relative significance to a survival for the sarcoma disease state;
determining whether a subject is a likely responder or a likely non-responder for the treatment based on the response score; and
administering a therapeutic dosage of the treatment to the patient if the subject is determined to be the likely responder.

39. (canceled)

40. The method of claim 38, wherein the administering of the treatment comprises:
administering a combination of durvalumab and tremelimumab to the patient.

41. The method of claim 40, wherein the administering of the treatment comprises any or more one of:
administering the durvalumab at a dosage of 1500 mg via an intravenous (IV) route of administration every 4 weeks for up to 4 doses;
administering the durvalumab at a dosage of 1500 mg via the IV route of administration every 4 weeks for up to 13 doses;
administering the durvalumab at a dosage of 1500 mg via the IV route of administration every 4 weeks starting at week 16 for up to 9 doses; and
administering the tremelimumab at a dosage of 75 mg via the IV route of administration every 4 weeks starting for up to 4 doses.

42.-73. (canceled)
